SEQUENCE LISTING (118 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 3 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 4 - protein, 227 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSVSHPYSQDLEGKGEWGP

SEQ ID NO: 5 - protein, 588 aa, Homo sapiens
MSSCVSSQPSSNRAAPQDELGGRGSSSSESQKPCEALRGLSSLSIHLGMESFIVVTECEPGCAVDLGLARDRPLEADGQEVPLDTSGSQARPHLSGRKLSLQERSQGGLAAGGSLDMNGRCICPSLPYSPVSSPQSSPRLPRRPTVESHHVSITGMQDCVQLNQYTLKDEIGKGSYGVVKLAYNENDNTYYAMKVLSKKKLIRQAGFPRRPPPRGTRPAPGGCIQPRGPIEQVYQEIAILKKLDHPNVVKLVEVLDDPNEDHLYMVFELVNQGPVMEVPTLKPLSEDQARFYFQDLIKGIEYLHYQKIIHRDIKPSNLLVGEDGHIKIADFGVSNEFKGSDALLSNTVGTPAFMAPESLSETRKIFSGKALDVWAMGVTLYCFVFGQCPFMDERIMCLHSKIKSQALEFPDQPDIAEDLKDLITRMLDKNPESRIVVPEIKLHPWVTRHGAEPLPSEDENCTLVEVTEEEVENSVKHIPSLATVILVKTMIRKRSFGNPFEGSRREERSLSAPGNLLTKKPTRECESLSELKEARQRRQPPGHRPAPRGGGGSALVRGSPCVESCWAPAPGSPARMHPLRPEEAMEPE

SEQ ID NO: 6 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 7 - protein, 1464 aa, Homo sapiens [sequence omitted]

SEQ ID NO: 8 - protein, 8 aa, Homo sapiens
GGEIEGFR

SEQ ID NO: 9 - protein, 16 aa, Homo sapiens
GLGTDEDSLIEIICSR

SEQ ID NO: 10 - protein, 11 aa, Homo sapiens
SLSAPGNLLTK

SEQ ID NO: 11 - protein, 11 aa, Homo sapiens
AYPDANLLNDR

SEQ ID NO: 12 - protein, 16 aa, Homo sapiens
LTQLGTFEDHFLSLQR

SEQ ID NO: 13 - protein, 16 aa, Homo sapiens
VIVPADPTLWSTDHVR

SEQ ID NO: 14 - protein, 14 aa, Homo sapiens
TYSVSFDSLFSAVK

SEQ ID NO: 15 - protein, 15 aa, Homo sapiens
LGLGADVAQVTGALR

SEQ ID NO: 16 - protein, 11 aa, Homo sapiens
DISEMFLQIYK

SEQ ID NO: 17 - protein, 13 aa, Homo sapiens
LLQDSSSPVDLAK

SEQ ID NO: 18 - protein, 11 aa, Homo sapiens
LSEPAETDAVK

SEQ ID NO: 19 - protein, 9 aa, Homo sapiens
YCQYAFDLK

SEQ ID NO: 20 - protein, 8 aa, Homo sapiens
QTSILIQK

SEQ ID NO: 21 - protein, 13 aa, Homo sapiens
LEAGDHPVELLAR

SEQ ID NO: 22 - protein, 11 aa, Homo sapiens
VDCGYPHVTPK

SEQ ID NO: 23 - protein, 7 aa, Homo sapiens
FMDVYQR

SEQ ID NO: 24 - protein, 18 aa, Homo sapiens
IPGVPWCFKPLQEAECTF

SEQ ID NO: 25 - protein, 11 aa, Homo sapiens
DEVAHTLTENR

SEQ ID NO: 26 - protein, 17 aa, Homo sapiens
FYGAEIVSALDYLHSEK

SEQ ID NO: 27 - protein, 10 aa, Homo sapiens
LQLGPEILQR

SEQ ID NO: 28 - protein, 12 aa, Homo sapiens
LSGFGQSGSFCR

SEQ ID NO: 29 - protein, 14 aa, Homo sapiens
ALLLFSIIPVDGLK

SEQ ID NO: 30 - protein, 14 aa, Homo sapiens
MLYFAPDLVFNEYR

SEQ ID NO: 31 - protein, 11 aa, Homo sapiens
SLYYYIQQDTK

SEQ ID NO: 32 - protein, 14 aa, Homo sapiens
VEFTFPDFVTEGAR

SEQ ID NO: 33 - protein, 10 aa, Homo sapiens
VLCPSNSSQR

SEQ ID NO: 34 - protein, 17 aa, Homo sapiens
AGFQTEDFSLYACASPK

SEQ ID NO: 35 - protein, 12 aa, Homo sapiens
LTQEHIEALLDK

SEQ ID NO: 36 - protein, 10 aa, Homo sapiens
SFGNPFEGSR

SEQ ID NO: 37 - protein, 13 aa, Homo sapiens
AEETCAPSVSYFK

SEQ ID NO: 38 - protein, 14 aa, Homo sapiens
DCDALMAGCIQEAR

SEQ ID NO: 39 - protein, 12 aa, Homo sapiens
LFGPVDSEQLSR

SEQ ID NO: 40 - protein, 8 aa, Homo sapiens
WANQCNYR

SEQ ID NO: 41 - protein, 9 aa, Homo sapiens
YEDLYSNCK

SEQ ID NO: 42 - protein, 14 aa, Homo sapiens
FEEITGVINPALDK

SEQ ID NO: 43 - protein, 16 aa, Homo sapiens
YYSSSIWGPTCDGLDR

SEQ ID NO: 44 - protein, 11 aa, Homo sapiens
NLQEILHGAVR

SEQ ID NO: 45 - protein, 13 aa, Homo sapiens
AIQTGEELFFDYR

SEQ ID NO: 46 - protein, 14 aa, Homo sapiens
IQPVHILTSVSSLR

SEQ ID NO: 47 - protein, 13 aa, Homo sapiens
IGPDNLPYVQILK

SEQ ID NO: 48 - protein, 13 aa, Homo sapiens
VYSDPQPHIQWLK

SEQ ID NO: 49 - protein, 17 aa, Homo sapiens
GVILYSDPADYFAPGVK

SEQ ID NO: 50 - protein, 12 aa, Homo sapiens
SPNVLSVALSQR

SEQ ID NO: 51 - protein, 14 aa, Homo sapiens
SSADPALNQEVALK

SEQ ID NO: 52 - protein, 10 aa, Homo sapiens
ALTHSELDVR

SEQ ID NO: 53 - protein, 12 aa, Homo sapiens
LLEVISVCDCPR

SEQ ID NO: 54 - protein, 17 aa, Homo sapiens
LATQSNEITIPVTFESR

SEQ ID NO: 55 - protein, 10 aa, Homo sapiens
LFDQAFGLPR

SEQ ID NO: 56 - protein, 13 aa, Homo sapiens
HFSTYGAAVAQNR

SEQ ID NO: 57 - protein, 19 aa, Homo sapiens
IYSTPFYSPQENVVFSSSR

SEQ ID NO: 58 - protein, 11 aa, Homo sapiens
LLCGATLIAPR

SEQ ID NO: 59 - protein, 12 aa, Homo sapiens
WLLTAAHCLKPR

SEQ ID NO: 60 - protein, 8 aa, Homo sapiens
NSQVWLGR

SEQ ID NO: 61 - protein, 17 aa, Homo sapiens
SLQCVSLHLLSNDMCAR

SEQ ID NO: 62 - protein, 12 aa, Homo sapiens
AFQVWSDVTPLR

SEQ ID NO: 63 - protein, 9 aa, Homo sapiens
VDAAFNWSK

SEQ ID NO: 64 - protein, 9 aa, Homo sapiens
FQTFEGDLK

SEQ ID NO: 65 - protein, 11 aa, Homo sapiens
NYGQLDIFPAR

SEQ ID NO: 66 - protein, 12 aa, Homo sapiens
VYHLPYYEACVR

SEQ ID NO: 67 - protein, 8 aa, Homo sapiens
GVLLWGWR

SEQ ID NO: 68 - protein, 14 aa, Homo sapiens
ATAYILSVQAEEQK

SEQ ID NO: 69 - protein, 17 aa, Homo sapiens
SGLCSPSYVAVTPFSLR

SEQ ID NO: 70 - protein, 19 aa, Homo sapiens
ATEYVHSLQAEEHQLLLEK

SEQ ID NO: 71 - protein, 13 aa, Homo sapiens
FELLPTPPLSPSR

SEQ ID NO: 72 - protein, 15 aa, Homo sapiens
IVEANPLLEAFGNAK

SEQ ID NO: 73 - protein, 13 aa, Homo sapiens
TVYSHLFDHVVNR

SEQ ID NO: 74 - protein, 10 aa, Homo sapiens
NFDGLEEIDR

SEQ ID NO: 75 - protein, 8 aa, Homo sapiens
HYINLITR

SEQ ID NO: 76 - protein, 13 aa, Homo sapiens
SSPETLISDLLMR

SEQ ID NO: 77 - protein, 10 aa, Homo sapiens
FGNSLHPIVR

SEQ ID NO: 78 - protein, 8 aa, Homo sapiens
TVLQLPSK

SEQ ID NO: 79 - protein, 17 aa, Homo sapiens
AIPVAQDLNAPSDWDSR

SEQ ID NO: 80 - protein, 16 aa, Homo sapiens
YPDAVATWLNPDPSQK

SEQ ID NO: 81 - protein, 15 aa, Homo sapiens
CSDSDGLAPPQHLIR

SEQ ID NO: 82 - protein, 10 aa, Homo sapiens
LGFLHSGTAK

SEQ ID NO: 83 - protein, 21 aa, Homo sapiens
IPQPLFFINSEYFQYPANIIK

SEQ ID NO: 84 - protein, 14 aa, Homo sapiens
YPLVVFSHGLGAFR

SEQ ID NO: 85 - protein, 15 aa, Homo sapiens
ELDIFGLNPADESTR

SEQ ID NO: 86 - protein, 11 aa, Homo sapiens
VVEGTAYGLSR

SEQ ID NO: 87 - protein, 9 aa, Homo sapiens
LINLTDILK

SEQ ID NO: 88 - protein, 11 aa, Homo sapiens
YEQYLDNLLVR

SEQ ID NO: 89 - protein, 9 aa, Homo sapiens
TISNGFGFK

SEQ ID NO: 90 - protein, 13 aa, Homo sapiens
VVDPTPEQFQAFR

SEQ ID NO: 91 - protein, 10 aa, Homo sapiens
ALGITEIFIK

SEQ ID NO: 92 - protein, 9 aa, Homo sapiens
NGEEFSFLK

SEQ ID NO: 93 - protein, 10 aa, Homo sapiens
CYNELNGCTK

SEQ ID NO: 94 - protein, 12 aa, Homo sapiens
APLIPMEHCTTR

SEQ ID NO: 95 - protein, 11 aa, Homo sapiens
EAVPEPVLLSR

SEQ ID NO: 96 - protein, 8 aa, Homo sapiens
VLLIETQR

SEQ ID NO: 97 - protein, 15 aa, Homo sapiens
VLTQASNPVVCTQPK

SEQ ID NO: 98 - protein, 15 aa, Homo sapiens
HIAEDSDRKYEEVAR

SEQ ID NO: 99 - protein, 11 aa, Homo sapiens
QLEEEQQALQK

SEQ ID NO: 100 - protein, 11 aa, Homo sapiens
MASCSYVAHER

SEQ ID NO: 101 - protein, 17 aa, Homo sapiens
YIDFLYQVLQSDELDSK

SEQ ID NO: 102 - protein, 11 aa, Homo sapiens
HLFVQDPQTCK

SEQ ID NO: 103 - protein, 9 aa, Homo sapiens
IVGGWECEK

SEQ ID NO: 104 - protein, 12 aa, Homo sapiens
LSEPAELTDAVK

SEQ ID NO: 105 - protein, 16 aa, Homo sapiens
TEMTASSSSDYGQTSK

SEQ ID NO: 106 - protein, 12 aa, Homo sapiens
MECNPSQVNGSR

SEQ ID NO: 107 - protein, 19 aa, Homo sapiens
MVGSPDTVGMNYGSYMEEK

SEQ ID NO: 108 - protein, 12 aa, Homo sapiens
HMPPPNMTTNER

SEQ ID NO: 109 - protein, 13 aa, Homo sapiens
ITTRPDLPYEPPR

SEQ ID NO: 110 - protein, 10 aa, Homo sapiens
NTDLPYEPPR

SEQ ID NO: 111 - protein, 6 aa, Homo sapiens
FSYGEK

SEQ ID NO: 112 - protein, 10 aa, Homo sapiens
QEGFLAHPSR

SEQ ID NO: 113 - protein, 9 aa, Homo sapiens
LIEPEEVAR

SEQ ID NO: 114 - protein, 8 aa, Homo sapiens
ITTRPGTK

SEQ ID NO: 115 - protein, 8 aa, Homo sapiens
ALSHVIQR

SEQ ID NO: 116 - protein, 9 aa, Homo sapiens
GFPHVIYAR

SEQ ID NO: 117 - protein, 9 aa, Homo sapiens
FPELNYQLK

SEQ ID NO: 118 - protein, 13 aa, Homo sapiens
GLSIEQLTTLAEK

(19) United States
(12) Patent Application Publication
(10) Pub. No.: US 2022/0334119 A1
(43) Pub. Date: Oct. 20, 2022

(54) PROTEIN PANELS FOR THE EARLY DIAGNOSIS/PROGNOSIS AND TREATMENT OF AGGRESSIVE PROSTATE CANCER

(71) Applicants: BATTELLE MEMORIAL INSTITUTE, Richland, WA (US); THE HENRY M. JACKSON FOUNDATION FOR THE ADVANCEMENT OF MILITARY MEDICINE, INC., BETHESDA, MD (US); THE UNITED STATES OF AMERICA,as represented by the Secretary,Department of Health and Human Services, Bethesda, MD (US)

(72) Inventors: Karin RODLAND, Richland, WA (US); Tao LIU, Richland, WA (US); Jennifer CULLEN, Bethesda, MD (US); Gyorgy PETROVICS, Bethesda, MD (US); Jacob KAGAN, Bethesda, MD (US); Sudhir SRIVASTAVA, Bethesda, MD (US)

(21) Appl. No.: 17/634,626

(22) PCT Filed: Aug. 19, 2020
(86) PCT No.: PCT/US2020/047069

§ 371 Date: Feb. 11, 2022

Related U.S. Application Data
(60) Provisional application No. 62/888,890, filed on Aug. 19, 2019.

(51) Int. Cl. G01N 33/574; G01N 33/68

(52) U.S. Cl. CPC G01N 33/57434; G01N 33/6848; G01N 2333/495; G01N 2333/948

(57) ABSTRACT

Disclosed herein are methods of diagnosing or prognosing aggressive prostate cancer in a subject and methods of treating a subject with aggressive prostate cancer. For example, the methods can include measuring increased expression of aggressive prostate cancer-related molecules (such as FOLH1, SPARC, TGFB1, CAMKK2, NCOA2, EGFR, or PSA) and optionally administering a therapeutically effective amount of aggressive prostate cancer therapy.

### GOVERNMENT INTEREST

This invention was made with government support under ACN15006-001 awarded by the National Institutes of Health. The government has certain rights in the invention.

### FIELD

This application provides methods of diagnosing and prognosing aggressive prostate cancer based on an increase in expression of aggressive prostate cancer-related molecules (such as FOLH1, SPARC, TGFB1, CAMKK2, NCOA2, EGFR, or combinations thereof), and in some examples also decreased expression of PSA. It also provides methods of treating aggressive prostate cancer based on the expression patterns of these aggressive prostate cancer-related molecules.

### SEQUENCE LISTING

The instant application contains a Sequence Listing which has been submitted electronically in ASCII format and is hereby incorporated by reference in its entirety. Said ASCII copy, created on Aug. 19, 2020, is named HMJ-172-PCT SL.txt and is 61,944 bytes in size.

### BACKGROUND

Prostate cancer (PCa) has a complex disease spectrum, ranging from clinically indolent to aggressive subtypes with a high degree of molecular and cellular heterogeneity. To provide personalized management of the disease, physicians and patients consider a wide variety of options both for determining the nature of the disease and then select the best treatment based clinical results.

However, prostate cancer screening based on serum prostate-specific antigen (PSA) results in many false positives, biopsy complications, and over-diagnosis that ultimately leads to overtreatment. Conventional repeat biopsies are inaccurate and pose unnecessary risks. Multi-parametric MRI (mpMRI) for initial detection and guiding biopsies in active surveillance improves risk stratification and identification of target lesions, but does not increase the overall rate of cancer detection. Previously, combining mpMRI and conventional biopsies has provided the highest detection rate.

Early detection of PCa in isolation is not sufficient to reduce mortality from the disease, as a large proportion of screen-detected lesions are indolent. Thus, there is a need to distinguish early between indolent and aggressive prostate cancer, for example, to select proper treatment and avoid over-treating indolent disease. Therefore, methods of assessing risk of aggressive prostate cancer with a clinically significant improvement in prognostic accuracy over current methods is desired.

### SUMMARY

Disclosed are methods of diagnosing or prognosing aggressive prostate cancer in a subject. Also disclosed herein are methods of treating a subject (such as a human subject) with aggressive prostate cancer. In some examples, the methods include measuring expression of aggressive prostate cancer-related molecules in a sample obtained from a subject, wherein the aggressive prostate cancer-related molecules include folate hydrolase 1 (FOLH1), secreted protein acidic and rich in cysteine (SPARC), and transforming growth factor beta 1 (TGFB1). In some examples, the methods include identifying the subject as having aggressive prostate cancer or at risk of developing aggressive prostate cancer if the sample from the subject contains increased expression of the aggressive prostate cancer-related molecules (and in some examples decreased expression of the aggressive prostate cancer-related molecule PSA) as compared to a control representing expression for the aggressive prostate cancer-related molecules expected in a sample from a subject who does not have aggressive prostate cancer. In some examples, if increased expression of the aggressive prostate cancer-related molecules is detected (and in some examples if decreased expression of the aggressive prostate cancer-related molecule PSA is also detected) in the sample, the methods include administering a therapeutically effective amount of aggressive prostate cancer therapy, thereby treating the subject.

In some embodiments of the methods of treatment, prior to the administration of the therapeutically effective amount of aggressive prostate cancer therapy, the expression level of the aggressive prostate cancer-related molecules in a sample from the subject was determined to be increased as compared to a control, wherein the control represents expression of the aggressive prostate cancer-related molecules expected in a sample from a subject who does not have aggressive prostate cancer, and wherein the aggressive prostate cancer-related molecules comprise folate hydrolase 1 (FOLH1), secreted protein acidic and rich in cysteine (SPARC), and transforming growth factor beta 1 (TGFB1).

In some embodiments, the aggressive prostate cancer-related molecules further include (a) one or more of calcium/calmodulin dependent protein kinase kinase 2 (CAMKK2), epidermal growth factor receptor (EGFR), nuclear receptor coactivator 2 (NCOA2), and prostate-specific antigen (PSA); (b) PSA; (c) CAMKK2, EGFR, and NCOA2; or (d) CAMKK2 and PSA. In embodiments, the subject with aggressive prostate cancer or at risk of developing aggressive prostate cancer includes a subject at risk for post-surgical biochemical recurrent prostate cancer.

In embodiments, the aggressive prostate cancer-related molecules further include PSA, wherein the subject with aggressive prostate cancer or at risk of developing aggressive prostate cancer includes a subject at risk for post-surgical distant metastatic prostate cancer. For example, the control representing expression for the aggressive prostate cancer-related molecule(s) expected in a sample from a subject who does not have aggressive prostate cancer includes a control representing expression for the aggressive prostate cancer-related molecule(s) expected in a sample from a subject who does not develop post-surgical distant metastatic prostate cancer.

In embodiments, the aggressive prostate cancer-related molecules further include CAMKK2, EGFR, and NCOA2, wherein the subject with aggressive prostate cancer has a prostate cancer with a high Gleason score or Grade Group. For example, the control representing expression for the aggressive prostate cancer-related molecule(s) expected in a sample from a subject who does not have prostate cancer can include a control representing expression for the aggressive prostate cancer-related molecule(s) expected in a sample from a subject who does not have prostate cancer with a high Gleason score or a high Grade Group.

In embodiments, the aggressive prostate cancer-related molecules further include CAMKK2 and PSA, wherein the subject with aggressive prostate cancer or at risk of developing aggressive prostate cancer includes a subject at risk for post-surgical distant metastatic prostate cancer, biochemical recurrent prostate cancer, or both. For example, the control representing expression for the aggressive prostate cancer-related molecule(s) expected in a sample from a subject who does not have aggressive prostate cancer can include a control representing expression for the aggressive prostate cancer-related molecule(s) expected in a sample from a subject who does not develop post-surgical biochemical recurrent prostate cancer. In some examples, the control representing expression for the aggressive prostate cancer-related molecule(s) expected in a sample from a subject who does not have aggressive prostate cancer includes a control representing expression for the aggressive prostate cancer-related molecule(s) expected in a sample from a subject who does not develop post-surgical distant metastatic prostate cancer.

In embodiments, the sample includes or is a prostatectomy sample, a biopsy sample (such as a fine needle aspirate), blood sample (such as a plasma or serum sample), urine, semen, or expressed prostatic secretion sample. In specific, non-limiting examples, the sample is a prostatectomy sample, such as a formalin-fixed paraffin-embedded (FFPE) sample.

In embodiments, expression of the aggressive prostate cancer-related molecules is determined using relative protein or peptide abundance. In examples, the abundance is determined using mass spectrometry, such as selected reaction monitoring (SRM). For example, the abundance can be determined using a high-pressure, high-resolution separations coupled with intelligent selection and multiplexing (PRISM)-SRM assay.

In embodiments, the subject has had a prostatectomy. In embodiments, the subject has had a prostatectomy, and the method further includes post-treatment surveillance.

In embodiments, the aggressive prostate cancer therapy administered to the subject includes at least one of surgery, radiation, hormone therapy, chemotherapy, brachytherapy, cryotherapy, ultrasound, bisphosphate therapy, biologic therapy, or vaccine therapy.

Further disclosed herein are methods of treating a sample obtained from a subject (such as a human subject) with a protease, thereby forming a digested sample. In examples, the methods can include measuring expression of (a) FOLH1, SPARC, and TGFB1; (b) FOLH1, SPARC, TGFB1, and CAMKK2; (c) FOLH1, SPARC, TGFB1, CAMKK2, EGFR, and NCOA2;(d) FOLH1, SPARC, TGFB1, CAMKK2, and PSA, or (e) FOLH1, SPARC, TGFB1, and PSA, in the digested sample using mass spectrometry.

In some embodiments of the diagnostic/prognostic and therapeutic methods, the aggressive prostate cancer-related molecules comprise folate hydrolase 1 (FOLH1), secreted protein acidic and rich in cysteine (SPARC), transforming growth factor beta 1 (TGFB1), calcium/calmodulin dependent protein kinase kinase 2 (CAMKK2), and prostate-specific antigen (PSA). In examples, the methods include measuring expression of aggressive prostate cancer-related molecules in a sample obtained from a subject, wherein the aggressive prostate cancer-related molecules include FOLH1, SPARC, TGFB1, CAMKK2 and PSA. In examples, the methods further include measuring increased expression of the aggressive prostate cancer-related molecules FOLH1, SPARC, TGFB1, CAMKK2, and decreased expression of the aggressive prostate cancer-related molecule PSA, as compared to a control representing expression for the aggressive prostate cancer-related molecule(s) expected in a sample from a subject who does not have aggressive prostate cancer. In examples, the methods include administering at least one of surgery, radiation, hormone therapy, chemotherapy, brachytherapy, cryotherapy, ultrasound, bisphosphate therapy, biologic therapy, or vaccine therapy to the subject with aggressive prostate cancer, thereby treating the subject. In embodiments, the subject has had a prostatectomy, and the methods further include post-treatment surveillance.

The foregoing and other objects and features of the disclosure will become more apparent from the following detailed description, which proceeds with reference to the accompanying figures.

### BRIEF DESCRIPTION OF THE DRAWINGS

FIGS. 1A-1D show ROC curves predicting distant metastasis or BCR using standard of care (SOC) base models and the protein panels versus SOC base models alone. Four proteins (FOLH1, PSA, SPARC, and TGFB1) were selected from univariable analysis and then formed a panel using PCA by selecting the PCs with Eigenvalue over 1 for predicting DM using the biopsy base model (FIG. 1A) and pathology base model (FIG. 1B); three proteins (FOLH1, SPARC, and TGFB1) were selected similarly for predicting BCR using the biopsy base model (FIG. 1C) and pathology base model (FIG. 1D). The biopsy base model included age at diagnosis, race, and NCCN risk stratum, and the pathology base model included age at diagnosis, race, pathological T stage, GG, and surgical margin. 95% CI of AUC was obtained using bootstrapped method with 1,000 replicate. The comparison between two AUCs using the SOC base models and the protein panels versus SOC base models alone was performed using maximum likelihood ratio test (P=0.002, 0.055, 0.003, and 0.050 in FIGS. 1A-1D, respectively).

FIGS. 2A-2D show Kaplan-Meier DM-free survival curves across high versus low groups for FOLH1 (FIG. 2A), PSA (FIG. 2B), SPARC (FIG. 2C), and TGFB1 (FIG. 2D).

FIGS. 3A-3B show Kaplan-Meier BCR-free survival curves across high versus low groups for SPARC (FIG. 3A) and TGFB1 (FIG. 3B).

FIGS. 4A-4B show the results for adding the 5-protein classifier (FOLH1, SPARC, TGFB1, CAMKK2, and PSA) to SOC to predict DM in a testing cohort.

FIGS. 5A-5P show response curves for the PRISM-SRM assays of 16 protein candidates. FIGS. 5A-5P disclose SEQ ID NOS 9-22, 8, and 23, respectively, in order of appearance.

FIGS. 6A-6B show interference in SRM detection of the heavy isotope-labeled internal standard for TGFB1 (GGEIEGFR (SEQ ID NO: 8)).

FIG. 7 shows whisker boxplots of 16 protein levels across event groups, showing 3 event groups for each protein (first, non-event; second, BCR; third, DM)

FIGS. 8A-8B show performance of 5-protein classifier (FOLH1, SPARC, TGFB1, CAMKK2, and PSA) in predicting DM among training (n=149) and testing (n=65) cohorts.

FIG. 9 shows Kaplan-Meier DM-free survival curves across dichotomized 5-protein classifier groups (high vs. low) in a testing cohort.

FIG. 10 shows Kaplan-Meier BCR-free survival curves across dichotomized 5-protein classifier groups (high vs. low) among a 285 BCR study cohort.

FIGS. 11A-11D show example peptides that can be used to measure aggressive prostate cancer-related molecules. FIGS. 11A-11D disclose SEQ ID NOS 24, 22, 25-30, 9, 31-36, 10, 37, 11, 38-43, 12, 44-49, 14, 50-64, 15-16, 65-73, 17, 74-93, 20, 94, 21, 95, 8, 96-101, 23, 102-108, 13, 109-116, 19, and 117-118, respectively, in order of appearance.

FIG. 12 shows a lower limit of detection (LOD) and quantification (LOQ) of example target proteins as well as slope and intercept values. FIG. 12 discloses SEQ ID NOS 9-22, 8, and 23, respectively, in order of appearance.

SEQUENCES

SEQ ID NO: 1 is an exemplary FOLH1 coding sequence:
MWNLLHETDSAVATARRPRWLCAGALVLAGGFFLLGFLFGWFIKSSNEATNITP
KHNMKAFLDELKAENIKKFLYNFTQIPHLAGTEQNFQLAKQIQSQWKEFGLDSV
ELAHYDVLLSYPNKTHPNYISIINEDGNEIFNTSLFEPPPPGYENVSDIVPPFSAFSP
QGMPEGDLVYVNYARTEDFFKLERDMKINCSGKIVIARYGKVFRGNKVKNAQL
AGAKGVILYSDPADYFAPGVKSYPDGWNLPGGGVQRGNILNLNGAGDPLTPGY
PANEYAYRRGIAEAVGLPSIPVHPIGYYDAQKLLEKMGGSAPPDSSWRGSLKVP
YNVGPGFTGNFSTQKVKMHIHSTNEVTRIYNVIGTLRGAVEPDRYVILGGHRDS
WVFGGIDPQSGAAVVHEIVRSFGTLKKEGWRPRRTILFASWDAEEFGLLGSTEW
AEENSRLLQERGVAYINADSSIEGNYTLRVDCTPLMYSLVHNLTKELKSPDEGFE
GKSLYESWTKKSPSPEFSGMPRISKLGSGNDFEVFFQRLGIASGRARYTKNWETN
KFSGYPLYHSVYETYELVEKFYDPMFKYHLTVAQVRGGMVFELANSIVLPFDCR
DYAVVLRKYADKIYSISMKHPQEMKTYSVSFDSLFSAVKNFTEIASKFSERLQDF
DKSNPIVLRMMNDQLMFLERAFIDPLGLPDRPFYRHVIYAPSSHNKYAGESFPGI
YDALFDIESKVDPSKAWGEVKRQIYVAAFTVQAAAETLSEVA
SEQ ID NO: 2 is an exemplary SPARC coding sequence:
MRAWIFFLLCLAGRALAAPQQEALPDETEVVEETVAEVTEVSVGANPVQVEVG
EFDDGAEETEEEVVAENPCQNHHCKHGKVCELDENNTPMCVCQDPTSCPAPIGE
FEKVCSNDNKTFDSSCHFFATKCTLEGTKKGHKLHLDYIGPCKYIPPCLDSELTEF
PLRMRDWLKNVLVTLYERDEDNNLLTEKQKLRVKKIHENEKRLEAGD
HPVELLARDFEKNYNMYIFPVHWQFGQLDQHPIDGYLSHTELAPLRAPLIPMEH
CTTRFFETCDLDNDKYIALDEWAGCFGIKQKDIDKDLVI
SEQ ID NO: 3 is an exemplary TGFB1 coding sequence:
MPPSGLRLLLLLLPLLWLLVLTPGRPAAGLSTCKTIDMELVKRKRIEAIRGQILSK
LRLASPPSQGEVPPGPLPEAVLALYNSTRDRVAGESAEPEPEPEADYYAKEVTRV
LMVETHNEIYDKFKQSTHSIYMFFNTSELREAVPEPVLLSRAELRLLRLKLKVEQ
HVELYQKYSNNSWRYLSNRLLAPSDSPEWLSFDVTGVVRQWLSRGGEIEGFRLS
AHCSCDSRDNTLQVDINGFTTGRRGDLATIHGMNRPFLLLMATPLERAQHLQSS
RHRRALDTNYCFSSTEKNCCVRQLYIDFRKDLGWKWIHEPKGYHANFCLGPCPY
IWSLDTQYSKVLALYNQHNPGASAAPCCVPQALEPLPIVYYVGRKPKVEQLSNM
IVRSCKCS
SEQ ID NO: 4 is an exemplary PSA coding sequence:
MWVPVVFLTLSVTWIGAAPLILSRIVGGWECEKHSQPWQVLVASRGRAVCGGV
LVHPQWVLTAAHCIRNKSVILLGRHSLFHPEDTGQVFQVSHSFPHPLYDMSLLK
NRFLRPGDDSSHDLMLLRLSEPAELTDAVKVMDLPTQEPALGTTCYASGWGSIE
PEEFLTPKKLQCVDLHVISNDVCAQVHPQKVTKFMLCAGRWTGGKSTCS
VSHPYSQDLEGKGEWGP
SEQ ID NO: 5 is an exemplary CAMKK2 coding sequence:
MSSCVSSQPSSNRAAPQDELGGRGSSSSESQKPCEALRGLSSLSIHLGMESFIVVT
ECEPGCAVDLGLARDRPLEADGQEVPLDTSGSQARPHLSGRKLSLQERSQGGLA
AGGSLDMNGRCICPSLPYSPVSSPQSSPRLPRRPTVESHHVSITGMQDCVQLNQY
TLKDEIGKGSYGVVKLAYNENDNTYYAMKVLSKKKLIRQAGFPRRPPPRGTRPA
PGGCIQPRGPIEQVYQEIAILKKLDHPNVVKLVEVLDDPNEDHLYMVFELVNQGP
VMEVPTLKPLSEDQARFYFQDLIKGIEYLHYQKIIHRDIKPSNLLVGEDGHIKIAD
FGVSNEFKGSDALLSNTVGTPAFMAPESLSETRKIFSGKALDVWAMGVTLYCFV
FGQCPFMDERIMCLHSKIKSQALEFPDQPDIAEDLKDLITRMLDKNPESRIVVPEI
KLHPWVTRHGAEPLPSEDENCTLVEVTEEEVENSVKHIPSLATVILVKTMIRKRS
FGNPFEGSRREERSLSAPGNLLTKKPTRECESLSELKEARQRRQPPGHRPAPRGG
GGSALVRGSPCVESCWAPAPGSPARMHPLRPEEAMEPE
SEQ ID NO: 6 is an exemplary EGFR coding sequence:
MRPSGTAGAALLALLAALCPASRALEEKKVCQGTSNKLTQLGTFEDHFLSLQRM
FNNCEVVLGNLEITYVQRNYDLSFLKTIQEVAGYVLIALNTVERIPLENLQIIRGN
MYYENSYALAVLSNYDANKTGLKELPMRNLQEILHGAVRFSNNPALCNVESIQ
WRDIVSSDFLSNMSMDFQNHLGSCQKCDPSCPNGSCWGAGEENCQKLTKIICAQ
QCSGRCRGKSPSDCCHNQCAAGCTGPRESDCLVCRKFRDEATCKDTCPPLMLY
NPTTYQMDVNPEGKYSFGATCVKKCPRNYVVTDHGSCVRACGADSYEMEEDG
VRKCKKCEGPCRKVCNGIGIGEFKDSLSINATNIKHFKNCTSISGDLHILPVAFRG
DSFTHTPPLDPQELDILKTVKEITGFLLIQAWPENRTDLHAFENLEIIRGRTKQHG
QFSLAVVSLNITSLGLRSLKEISDGDVIISGNKNLCYANTINWKKLFGTSGQKTKII
SNRGENSCKATGQVCHALCSPEGCWGPEPRDCVSCRNVSRGRECVDKCKLLEG
EPREFVENSECIQCHPECLPQAMNITCTGRGPDNCIQCAHYIDGPHCVKTCPAGV
MGENNTLVWKYADAGHVCHLCHPNCTYGCTGPGLEGCPTNGPKIPSIATGMVG
ALLLLLVVALGIGLFMRRRHIVRKRTLRRLLQERELVEPLTPSGEAPNQALLRILK
ETEFKKIKVLGSGAFGTVYKGLWIPEGEKVKIPVAIKELREATSPKANKEILDEAY
VMASVDNPHVCRLLGICLTSTVQLITQLMPFGCLLDYVREHKDNIGSQYLLNWC
VQIAKGMNYLEDRRLVHRDLAARNVLVKTPQHVKITDFGLAKLLGAEEKEYHA
EGGKVPIKWMALESILHRIYTHQSDVWSYGVTVWELMTFGSKPYDGIPASEISSI
LEKGERLPQPPICTIDVYMIMVKCWMIDADSRPKFRELIIEFSKMARDPQRYLVIQ
GDERMHLPSPTDSNFYRALMDEEDMDDVVDADEYLIPQQGFFSSPSTSRTPLLSS
LSATSNNSTVACIDRNGLQSCPIKEDSFLQRYSSDPTGALTEDSIDDTFLPVPEYIN
QSVPKRPAGSVQNPVYHNQPLNPAPSRDPHYQDPHSTAVGNPEYLNTVQPTCVN
STFDSPAHWAQKGSHQISLDNPDYQQDFFPKEAKPNGIFKGSTAENAEYLRVAP
QSSEFIGA
SEQ ID NO: 7 is an exemplary NCOA2 coding sequence:
MSGMGENTSDPSRAETRKRKECPDQLGPSPKRNTEKRNREQENKYIEELAELIFA
NFNDIDNFNFKPDKCAILKETVKQIRQIKEQEKAAAANIDEVQKSDVSSTGQGVI
DKDALGPMMLEALDGFFFVVNLEGNVVFVSENVTQYLRYNQEELMNKSVYSIL
HVGDHTEFVKNLLPKSIVNGGSWSGEPPRRNSHTFNCRMLVKPLPDSEEEGHDN
QEAHQKYETMQCFAVSQPKSIKEEGEDLQSCLICVARRVPMKERPVLPSSESFTT
RQDLQGKITSLDTSTMRAAMKPGWEDLVRRCIQKFHAQHEGESVSYAKRHHHE
VLRQGLAFSQIYRFSLSDGTLVAAQTKSKLIRSQTTNEPQLVISLHMLHREQNVC
VMNPDLTGQTMGKPLNPISSNSPAHQALCSGNPGQDMTLSSNINFPINGPKEQM
GMPMGRFGGSGGMNHVSGMQATTPQGSNYALKMNSPSQSSPGMNPGQPTSML
SPRHRMSPGVAGSPRIPPSQFSPAGSLHSPVGVCSSTGNSHSYTNSSLNALQALSE
GHGVSLGSSLASPDLKMGNLQNSPVNMNPPPLSKMGSLDSKDCFGLYGEPSEGT
TGQAESSCHPGEQKETNDPNLPPAVSSERADGQSRLHDSKGQTKLLQLLTTKSD
QMEPSPLASSLSDTNKDSTGSLPGSGSTHGTSLKEKHKILHRLLQDSSSPVDLAKL
TAEATGKDLSQESSSTAPGSEVTIKQEPVSPKKKENALLRYLLDKDDTKDIGLPEI
TPKLERLDSKTDPASNTKLIAMKTEKEEMSFEPGDQPGSELDNLEEILDDLQNSQ
LPQLFPDTRPGAPAGSVDKQAIINDLMQLTAENSPVTPVGAQKTALRISQSTFNN
PRPGQLGRLLPNQNLPLDITLQSPTGAGPFPPIRNSSPYSVIPQPGMMGNQGMIGN
QGNLGNSSTGMIGNSASRPTMPSGEWAPQSSAVRVTCAATTSAMNRPVQGGMI
RNPAASIPMRPSSQPGQRQTLQSQVMNIGPSELEMNMGGPQYSQQQAPPNQTAP
WPESILPIDQASFASQNRQPFGSSPDDLLCPHPAAESPSDEGALLDQLYLALRNFD
GLEEIDRALGIPELVSQSQAVDPEQFSSQDSNIMLEQKAPVFPQQYASQAQMAQG
SYSPMQDPNFHTMGQRPSYATLRMQPRPGLRPTGLVQNQPNQLRLQLQHRLQA
QQNRQPLMNQISNVSNVNLTLRPGVPTQAPINAQMLAQRQREILNQHLRQRQM
HQQQQVQQRTLMMRGQGLNMTPSMVAPSGIPATMSNPRIPQANAQQFPFPPNY
GISQQPDPGFTGATTPQSPLMSPRMAHTQSPMMQQSQANPAYQAPSDINGWAQ
GNMGGNSMFSQQSPPHFGQQANTSMYSNNMNINVSMATNTGGMSSMNQMTG
QISMTSVTSVPTSGLSSMGPEQVNDPALRGGNLFPNQLPGMDMIKQEGDTTRKY
C

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising an aggressive prostate cancer-related molecule” includes single or plural molecules and is considered equivalent to the phrase “comprising at least one aggressive prostate cancer-related molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. or UniProt® Entry IDs referred to herein are the sequences available at least as early as Aug. 19, 2019. All references, including journal articles, patents, and patent publications, and GenBank® Accession numbers cited herein are incorporated by reference in their entirety.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Administration/delivery: To provide or give a subject an agent or therapy by any effective route, for example, administration of prostate cancer therapy (such as aggressive prostate cancer therapy). Examples of agents include chemotherapy, surgery, radiation therapy, targeted therapy (such as bisphosphonate therapy or hormone therapy), biologic therapy (such as immunotherapy or vaccine therapy), brachytherapy, cryotherapy, ultrasound, or palliative care. Administration further includes acute and chronic administration as well as local and systemic administration. In some examples, administration of a therapeutic agent, such as chemotherapy, is by injection (e.g., intravenous, intramuscular, intraosseous, intratumoral, intraprostatic, or intraperitoneal). In some examples, administration therapeutic agent, such as chemotherapy, is oral, transdermal, or rectal. In some examples, therapy includes active surveillance, such as post-treatment surveillance.

Animal: Living multi-cellular vertebrate organisms, a category that includes, for example, mammals and birds. The term mammal includes both human and non-human mammals. Similarly, the term “subject” includes both human and veterinary subjects.

Biochemical Recurrence (BCR): Also known as prostate-specific antigen (PSA) failure or biochemical relapse after a prostatectomy, a variety of factors can indicate BCR (see, for example, Tourinho-Barbosa et al., Int Braz J Urol., 44 (1): 14-21, 2018, incorporated herein by reference in its entirety). In an example, a PSA level of about 0.1 ng/mL to about 0.5 or about ≥0.2 ng/mL or ≥0.4 ng/mL. Additional factors include Gleason score (GS) or Grad Group (GG), PSA doubling time (PSA-DT), clinical stage, and surgical margins status.

Calcium/Calmodulin Dependent Protein Kinase Kinase 2 (CAMKK2): Also known as calcium/calmodulin dependent protein kinase kinase beta (CAMKKB) and KIAA0787, such as OMIM no. 615002, CAMKK2 is a kinase that regulates production of the appetite stimulating hormone neuropeptide Y and functions as an AMPK kinase in the hypothalamus. CAMKK2 nucleic acids and proteins are included. Exemplary CAMKK2 proteins, mRNA, and DNA include GENBANK® sequences NP_006540.3, BC026060.2, and AH010868.3, respectively. Other CAMKK2 molecules are possible. One of ordinary skill in the art can identify additional human, mouse, and rat CAMKK2 nucleic acid and protein sequences, including CAMKK2 variants that retain biological activity (such as kinase activity). In some examples, CAMKK2 is upregulated in a subject with prostate cancer, such as aggressive prostate cancer.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Chemotherapeutic agent or Chemotherapy: Any chemical or biological agent (such as a monoclonal antibody, mAb) with therapeutic usefulness in the treatment of diseases characterized by abnormal cell growth. Such diseases include tumors, neoplasms, and cancer, including prostate cancer. In one embodiment, a chemotherapeutic agent is an agent of use in treating aggressive prostate cancer. In some examples, chemotherapeutic agents used in the disclosed methods include cabazataxel (Jevtana®), docetaxel (Taxotere®), mitoxantrone (Teva®), or androgen deprivation therapy (ADT), such as with abiraterone Acetate (Zytiga®), bicalutamide (Casodex®), buserelin Acetate (Suprefact®), cyproterone Acetate (Androcur®), degarelix Acetate (Firmagon®), enzalutamide (Xtandi®), flutamide (Euflex®), goserelin Acetate (Zoladex®), histrelin Acetate (Vantas®), leuprolide Acetate (Lupron®, Eligard®), triptorelin Pamoate (Trelstar®). The therapy used in the disclosed methods can also include drugs to treat bone metastases (bisphosphate therapy), such as alendronate (Fosamax®), denosumab (Xgeva®), pamidronate (Aredia®), zoledronic acid (Zometa®), or radiopharmaceuticals, such as radium 223 (Xofigo®), strontium-89 (Metastron®), and samarium-153 (Quadramet®). Exemplary chemotherapeutic agents that can be used with the disclosed methods are provided in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993, all incorporated herein by reference. Combination chemotherapy is the administration of more than one agent (such as more than one chemical chemotherapeutic agent) to treat cancer. Such a combination can be administered simultaneously, contemporaneously, or with a period of time in between.

Control: A reference standard. In some embodiments, the control is a healthy subject. In other embodiments, the control is a subject with a cancer, such as a prostate cancer. In some embodiments, the control is a subject who does not have aggressive prostate cancer. In still other embodiments, the control is a historical control or standard reference value or range of values (e.g., a previously tested control subject with a known prognosis or outcome or group of subjects that represent baseline or normal values). A difference between a test subject and a control can be an increase or a decrease, such as an increase in expression of aggressive prostate cancer-related molecules. The difference can be a qualitative difference or a quantitative difference, for example a statistically significant difference.

Detect: To determine if an agent (such as a signal; particular nucleotide; amino acid; nucleic acid molecule and/or nucleotide modification; peptide or protein, such as a protein or peptide thereof of aggressive prostate cancer-related molecules; and/or organism) is present or absent. In some examples, detection can include further quantification. For example, use of the disclosed methods in particular examples permits detection or quantification of a protein or peptide thereof of aggressive prostate cancer-related molecules in a sample.

Differential Expression: A nucleic acid molecule is differentially expressed when the amount of one or more of its expression products (e.g., transcript and/or protein or peptide) is higher or lower in one sample (such as a test sample) as compared to another sample (such as a control). Detecting differential expression can include measuring a change in gene or protein (such as by measuring peptides thereof) expression.

Distant Metastasis (DM): Distant metastasis means that prostate cancer has spread beyond the original tumor, such as an original prostate tumor in the pelvis, such as to bone, spine, brain, liver, or lungs. Prostate cancer with distant metastasis has a 5-year survival rate of about 29 percent.

Downregulated or knocked down: When used in reference to the expression of a molecule, such as PSA RNA or protein, refers to any process which results in a decrease in production of PSA, but in some examples not complete elimination of PSA. In one example, downregulation or knock down does not result in complete elimination of detectable PSA expression or PSA activity.

Downregulation or knock down includes any detectable decrease in PSA. In certain examples, detectable PSA in a sample decreases by at least 10%, at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of PSA detected in a corresponding control representing PSA expression expected in a sample from a subject who does not have aggressive prostate cancer).

Epidermal growth factor receptor (EGFR): Also known as V-ERB-B avian erythroblastic leukemia viral oncogene homolog, oncogene ERBB, ERBB, ERBB1, HER1, NISBD2, PIG61, mENA, and species antigen 7 (SA7), such as OMIM no. 131550 is a receptor for epidermal growth factors (extracellular protein ligands). EGFR nucleic acids and proteins are included. Exemplary EGFR proteins, mRNA, and DNA include GENBANK® sequences CAA25240.1, M34309.1, and AF040717.1, respectively. Other EGFR molecules are possible. One of ordinary skill in the art can identify additional human, mouse, and rat EGFR nucleic acid and protein sequences, including EGFR variants that retain biological activity (such as receptor activity). In some examples, EGFR is upregulated in a subject with prostate cancer, such as aggressive prostate cancer.

Expression: Translation of a nucleic acid into a peptide or protein. Peptides or proteins may be expressed and remain intracellular, become a component of the cell surface membrane, or be secreted into the extracellular matrix or medium.

Folate hydrolase 1 (FOLH1): Also known as glutamate carboxypeptidase II (GCP2), prostate-specific membrane antigen (PSM or PSMA), N-acetylated alpha-linked acidic dipeptidase 1 (NAALAD1 or NAALADase I), such as OMIM no. 600934, FOLH1 is a membrane-associated zinc metalloenzyme. FOLH1 nucleic acids and proteins are included. Exemplary FOLH1 proteins, mRNA, and DNA include GENBANK® sequences NP 004467.1, NM 004476.3, and NG 029170.1, respectively. Other FOLH1 molecules are possible. One of ordinary skill in the art can identify additional human, mouse, and rat FOLH1 nucleic acid and protein sequences, including FOLH1 variants that retain biological activity (such as metalloprotease activity). In some examples, FOLH1 is upregulated in a subject with prostate cancer, such as aggressive prostate cancer.

Inhibiting or treating a disease: Inhibiting the full development of a disease or condition, for example, in a subject who is at risk for a disease, such as a subject with cancer, for example, prostate cancer (such as aggressive prostate cancer). “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Label: An agent capable of detection, for example by mass spectrometry, ELISA, spectrophotometry, flow cytometry, or microscopy. For example, a label can be attached to a nucleic acid molecule or protein, thereby permitting detection of the nucleic acid molecule or protein. For example, a protein or peptide can be produced as a heavy, stable isotope, but as a protein or peptide with ¹³C or ¹⁵N incorporated as a heavy, stable isotope. Examples of labels include, but are not limited to, radioactive or heavy, stable isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Nuclear receptor coactivator 2 (NCOA2): Also known as glucocorticoid receptor-interacting protein 1 (GRIP1), transcriptional intermediary factor 2 (TIF2), p160 steroid receptor coactivator 2 (SRC2), KAT13C, NCoA-2, and bHLHe75, such as OMIM no. 601993, NCOA2 is s a transcriptional coregulatory protein that contains several nuclear receptor interacting domains and has histone acetyltransferase activity. NCOA2 nucleic acids and proteins are included. Exemplary NCOA2 proteins, mRNA, and DNA include GENBANK® sequences AAI14384.1, BC114383.1, and NG_021400.2, respectively. Other NCOA2 molecules are possible. One of ordinary skill in the art can identify additional human, mouse, and rat NCOA2 nucleic acid and protein sequences, including NCOA2 variants that retain biological activity (such as histone acetyltransferase activity). In some examples, NCOA2 is upregulated in a subject with prostate cancer, such as aggressive prostate cancer.

Prognosis or Prognosing: The terms “prognosis” and “prognosing” as used herein mean predicting the likelihood of death from the cancer and/or recurrence or metastasis of the cancer within a given time period or predicting the likelihood of developing cancer during the patient's lifetime, with or without consideration of the likelihood that the cancer patient will respond favorably or unfavorably to a chosen therapy or therapies.

Prostate cancer: Also known as carcinoma of the prostate, prostate cancer is the development of cancer in the prostate, a gland in the male reproductive system. Prostate cancer can be considered aggressive or indolent. A variety of features can indicate aggressive prostate cancer, such as risk categories, tumor scoring or grouping, and patient events. Examples of risk categories include low-, intermediate-, and high-risk prostate cancer, which means that a patient has a low-, intermediate-, and high-risk, respectively, of pathological and biochemical outcomes after radical prostatectomy; metastasis; prostate cancer-specific mortality; and all-cause mortality (Cooperberg et al., J Cancer Inst., 101 (12):878-887, 2009). Another means of assessing the risk is using Gleason scoring or Grade Groups (GG; see Gordetsky and Epstein Diagn Pathol, 11:25, 2016, incorporated herein by reference in its entirety): very low- and low-risk prostate cancer, Gleason score sum less than or equal to 6 (GG 1); intermediate-risk prostate cancer, Gleason score sum at 7 (GG 2-3); and high-risk prostate cancer, Gleason score sum greater than 7 (GG 4-5). Examples of events that indicate aggressive prostate cancer include biochemical recurrence or distant metastasis, such as after a prostatectomy.

Most prostate cancers are slow growing; however, some grow relatively quickly. The cancer cells may spread from the prostate to other parts of the body, particularly the bones and lymph nodes. It may initially cause no symptoms. In later stages, it can lead to difficulty urinating, blood in the urine, or pain in the pelvis, back or when urinating or to feeling tired due to low levels of red blood cells. Prostate cancer can be diagnosed by biopsy. Medical imaging may then be done to determine if the cancer has spread to other parts of the body. Prostate cancer screening is controversial. Prostate-specific antigen PSA) testing increases cancer detection but does not decrease mortality. The United States Preventive Services Task Force recommends against screening using the PSA test, due to the risk of overdiagnosis and overtreatment, as most cancer diagnosed would remain asymptomatic, and concludes that the potential benefits of testing do not outweigh the expected harms.

Many cases can be safely followed with active surveillance (for example, after a prostatectomy) or watchful waiting. Other treatments, such as for aggressive prostate cancer, may include a combination of surgery (such as cryotherapy), radiation therapy, hormone therapy, and chemotherapy. When it only occurs inside the prostate, it may be curable. In those in whom the disease has spread to the bones, pain medications, bisphosphonates and targeted therapy, among others, may be useful. Outcomes depend on a person's age and other health problems as well as how aggressive and extensive the cancer is. Most people with prostate cancer do not die from the disease. The 5-year survival rate in the United States is 99%. Globally, it is the second most common type of cancer and the fifth leading cause of cancer-related death in men. Studies of males who died from unrelated causes have found prostate cancer in 30% to 70% of those over age 60.

Prostate-specific antigen (PSA): Also known as kallikrein-related peptidase 3 (kallikrein 3, KLK3; e.g., OMIM 176820); antigen, prostate-specific (APS); and gamma-seminoprotein, PSA is a glycoprotein and member of the kallikrein-related peptidase family. PSA is predominantly secreted by epithelial cells in the prostate gland and functions to dissolve cervical mucus to facilitate sperm entry into the uterus. PSA has been used to diagnose prostate cancer, as increased PSA levels in blood may suggest the presence of prostate cancer.

Includes PSA nucleic acid molecules and proteins. PSA sequences are publicly available. Nucleic acid and protein sequences for PSA are publicly available. For example, GenBank® Accession Nos. NM_001648.2, NM_012725.2, and NM_008455.3 discloses exemplary human PSA nucleotide sequences, respectively, and GenBank® Accession Nos. CAD54617.1, AAH89815.1, and NP_001639.1 discloses exemplary human PSA protein sequences. One of ordinary skill in the art can identify additional

PSA nucleic acid and protein sequences, including PSA variants that retain PSA biological activity (such as being secreted by the prostate gland).

Sample or biological sample: A sample of biological material obtained from a subject, which can include cells, proteins, and/or nucleic acid molecules. Biological samples include all clinical samples useful for detection of disease, such as cancer (including prostate cancer, for example, aggressive prostate cancer), in subjects. Appropriate samples include any conventional biological samples, including clinical samples obtained from a human or veterinary subject. Exemplary samples include, without limitation, cancer samples (such as from surgery, tissue biopsy, tissue sections, or autopsy, for example, a prostatectomy sample, such as a formalin-fixed paraffin-embedded (FFPE) sample), cells, cell lysates, blood smears, cytocentrifuge preparations, cytology smears, bodily fluids (e.g., blood, plasma, serum, saliva, sputum, urine, bronchoalveolar lavage, semen or expressed prostatic secretion, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as prostate cancer, for example, aggressive prostate cancer). In a specific example, the sample is a prostate cancer sample. In another specific example, the sample is a colorectal cancer sample.

Secreted Protein Acidic and Rich in Cysteine (SPARC): Also known as OSTEONECTIN (ON), BM40, and 0117, such as OMIM no. 182120, SPARC2 is a glycoprotein that binds calcium and has an affinity for collagen. SPARC2 nucleic acids and proteins are included. Exemplary SPARC2 proteins, mRNA, and DNA include GENBANK® sequences CAG33080.1, CR456799.1, and AY418497.1, respectively. Other SPARC2 molecules are possible. One of ordinary skill in the art can identify additional human, mouse, and rat SPARC2 nucleic acid and protein sequences, including SPARC2 variants that retain biological activity (such as calcium-binding activity). In some examples, SPARC2 is upregulated in a subject with prostate cancer, such as aggressive prostate cancer.

Subject: Living multi-cellular vertebrate organisms, a category that includes mammals, such as human and non-human mammals, such as veterinary subjects (for example cats, dogs, cows, sheep, horses, pigs, and mice). In a particular example, a subject is one who has or is at risk for aggressive prostate cancer, such as intermediate-, or high-risk prostate cancer. In a particular example, a subject is one who is suspected of having aggressive prostate cancer. In additional examples, a subject is one who has undergone a prostatectomy, such as a post-surgical subject suspected of having aggressive prostate cancer.

Transforming Growth Factor Beta 1 (TGFB1): Also known as transforming growth factor beta 1 induced transcript 1 (TGFB1I1), 55-KD transforming growth factor beta-induced, androgen receptor coactivator (ARA55), and HICS, such as OMIM no. 602353, TGFB1 is a cytokine in the transforming growth factor superfamily, the activated form of which complexes with other factors to form a serine/threonine kinase complex that binds TGF-β receptors. SPARC2 nucleic acids and proteins are included. Exemplary TGFB1 proteins, mRNA, and DNA include GENBANK® sequences AAH00125.1, NM_000660.7, and DQ309025.1, respectively. Other TGFB1 molecules are possible. One of ordinary skill in the art can identify additional human, mouse, and rat TGFB1 nucleic acid and protein sequences, including TGFB1 variants that retain biological activity (such as complex forming- or receptor-binding activity). In some examples, TGFB1 is upregulated in a subject with prostate cancer, such as aggressive prostate cancer.

Therapeutically effective amount: The amount of an active ingredient (such as a chemotherapeutic agent) that is sufficient to effect treatment when administered to a mammal in need of such treatment, such as treatment of a cancer. The therapeutically effective amount can vary depending upon the subject and disease condition being treated, the weight and age of the subject, the severity of the disease condition, the manner of administration and the like, which can readily be determined by a prescribing physician.

Treating, treatment, and therapy: Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's sensorimotor function. The treatment may be assessed by objective or subjective parameters; including the results of a physical examination, neurological examination, or psychiatric evaluations. For example, treatment of a cancer can include decreasing the size, volume, or weight of a cancer (such as a decrease of at least 10%, at least 20%, at least 50%, at least 75%, at least 80%, at least 85%, at least 90%, at least 95%, at least 98%, at least 99% or even 100%, for example as compared to no treatment, such as treatment with the disclosed methods), decrease the number, size, volume, or weight of metastases (such as a decrease of at least 10%, at least 20%, at least 50%, at least 75%, at least 80%, at least 85%, at least 90%, at least 95%, at least 98%, at least 99% or even 100%, for example as compared to no treatment, such as treatment with the disclosed methods), or combinations thereof. In specific examples, treatment or therapy can include chemotherapy, surgery, radiation therapy, targeted therapy (such as bisphosphonate therapy or hormone therapy), biologic therapy (such as immunotherapy or vaccine therapy), brachytherapy, cryotherapy, ultrasound, palliative care, or active surveillance (such as after a prostatectomy).

Tumor, neoplasia, malignancy or cancer: A neoplasm is an abnormal growth of tissue or cells which results from excessive cell division. Neoplastic growth can produce a tumor. The amount of a tumor in an individual is the “tumor burden”, which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer. Exemplary tumors, such as cancers, that can be analyzed and treated with the disclosed methods include prostate cancer, such as aggressive prostate cancer.

Upregulated or activation: When used in reference to the expression of a nucleic acid molecule, such as a gene, refers to any process which results in an increase in the production of a gene product. A gene product can be RNA (such as mRNA, rRNA, tRNA, and structural RNA) or protein. Therefore, gene upregulation or activation includes processes that increase transcription of a gene or translation of mRNA, such as FOLH1, SPARC, TGFB1, CAMKK2, EGFR, or NCOA2.

Examples of processes that increase transcription include those that facilitate formation of a transcription initiation complex, those that increase transcription initiation rate, those that increase transcription elongation rate, those that increase processivity of transcription, and those that relieve transcriptional repression (for example, by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product, such as a protein (e.g., FOLH1, SPARC, TGFB1, CAMKK2, EGFR, or NCOA2). In certain examples, production of a gene product increases by at least 2-fold, at least 3-fold, at least 4-fold, or at least 5-fold, as compared to a control (e.g., as compared to a threshold of expression of any of these molecules established from a subject or subjects, such as a cohort of control subjects, such as a control representing expression for FOLH1, SPARC, TGFB1, CAMKK2, EGFR, or NCOA2 expected in a sample from a subject who does not have aggressive prostate cancer).

### Overview

Disclosed herein are methods of measuring protein expression of low-abundance proteins using mass spectrometry (MS)-based targeted proteomics analysis of formalin-fixed and paraffin embedded (FFPE) specimens from organ-confined primary prostate tumors, in which differential protein abundance is used to identify proteins associated with prostate cancer aggressiveness, and the predictive accuracy of a robust protein marker subset is validated for local and distant cancer progression in a cohort of men with long-term follow-up data and detailed clinical annotation. Adding the proteomic classifier to the traditional biopsy-based prognostic model improved the area under the receiver operating curve (AUROC) by 0.2 units, achieving an AUC of 0.92. In some examples, the disclosed methods have a sensitivity of at least 90%. In some examples, the disclosed methods have a specificity of at least 50%.

### Evaluating Expression in a Subject With a Risk of Aggressive Prostate Cancer

Disclosed herein are methods of treating a subject (such as a human subject) with aggressive prostate cancer or at risk of developing aggressive prostate cancer. In some embodiments, the subject has had a prostatectomy, for example, the subject can have had a prostatectomy with post-treatment surveillance (such as active surveillance). In particular examples, the methods can determine with high specificity, sensitivity, or accuracy. For example, the sensitivity (such as measured using a negative predictive value) can be of at least 75%, at least 80%, at least 85%, at least 90%, at least 92%, at least 95%, at least 97%, at least 98%, or at least 99%, or about 75%-99%, about 75%-90%, about 85%-99%, about 90%-99%, about 95%-99%, about 97%-99%, such as a sensitivity of at least 90% or at least 92%. In some examples, the specificity can be least 35%, at least 40%, at least 45%, at least 50%, at least 55%, at least 60%, at least 65%, at least 70%, at least 75%, at least 80%, at least 90% or at least 99%, or about 35%-99%, about 35%-65%, about 40%-60%, about 50%-60%, about 50%-75%, or about 50%-90%, such as a specificity of at least 50% or at least 53%.

The subject can have a variety of aggressive prostate cancer features, such as biochemical recurrence (BCR), distant metastasis (DM), or a high Gleason score (GS, such as about 7 (for example, the Gleason sum is 3+4 or 4+3), 8, 9, or 10, or about 7-10 or 8-10) or high Group Grade (GG, such as about 3, 4, or 5, or about 3-5 or 4-5). It is helpful to determine whether or not a subject has an aggressive (or non-aggressive) prostate cancer because there are a variety of protocols for diagnosing prostate cancer, but not all are specific, sensitive, or accurate. Hence, using the results of the disclosed assays to help distinguish subjects that are likely to have aggressive prostate cancer versus those not likely to have aggressive prostate cancer offers a substantial clinical benefit and allows subjects to be accurately diagnosed and, if a subject has aggressive prostate cancer, to be accurately treated. Alternatively, if a subject does not have an aggressive prostate cancer, instead of additional treatment, the subject can be monitored (e.g., watchful waiting).

In additional examples, the methods are utilized to determine whether or not to provide the subject with therapeutic intervention. In one example, a therapeutic intervention is administered. Thus, if the subject has aggressive prostate cancer, a therapeutic intervention, such as surgery, radiation, hormone therapy, chemotherapy, brachytherapy, cryotherapy, ultrasound, bisphosphate therapy, biologic therapy, or vaccine therapy can be utilized. Using the results of the disclosed assays to help distinguish subjects that are likely to have aggressive prostate cancer versus those not likely to have aggressive prostate cancer offers a substantial clinical benefit because, where the subject has aggressive prostate cancer, the methods disclosed herein allow the subject to be selected for therapeutic intervention.

Methods of treating a subject with a risk of aggressive prostate cancer, such as with BRC, DM, or a high GS or high GG, are provided. Such methods can include measuring expression of aggressive prostate cancer-related molecules in a sample (such as a prostatectomy sample) obtained from a subject. For example, such methods can include measuring or detecting the absolute or relative amounts of aggressive prostate cancer-related molecules in the sample, such as aggressive prostate cancer-related proteins or peptides thereof or antibodies, nucleic acid probes, or nucleic acid primers specific for aggressive prostate cancer-related molecules. In some examples, the aggressive prostate cancer-related molecules can include at least about 3, 4, 5, 6, or 7 of folate hydrolase 1 (FOLH1), secreted protein acidic and rich in cysteine (SPARC), transforming growth factor beta 1 (TGFB1), calcium/calmodulin dependent protein kinase kinase 2 (CAMKK2), epidermal growth factor receptor (EGFR), nuclear receptor coactivator 2 (NCOA2), or prostate-specific antigen (PSA). The expression levels of these molecules can be measured. If increased protein or nucleic acid expression of FOLH1, SPARC, and TGFB1 (and in some examples also 1, 2 or 3 of CAMKK2, NCOA2, and EGFR), and in some examples also decreased protein or nucleic acid expression of PSA, in the sample is measured, the methods can include administering therapeutic intervention to the subject, thereby treating the subject.

Further disclosed herein are methods of treating a sample obtained from a subject (such as a human subject) with a protease (such as trypsin, but other proteases are possible, see, for example, Giansanti et al., Nat Protoc., 11 (5):993-1006, 2016), thereby forming a digested sample. The expression levels of peptide from the digested samples can be measured. In examples, the methods can include measuring expression of peptides from at least about 3, 4, 5, 6, or 7 of FOLH1, SPARC, TGFB1, CAMKK2, NCOA2, EGFR, or PSA proteins, for example, using mass spectroscopy. In specific, non-limiting examples, combinations of peptide from these proteins can be measured. For example, peptides from the combination FOLH1, SPARC, and TGFB1 can be measured. In some embodiments, peptides from the combination FOLH1, SPARC, TGFB1, and CAMKK2 can be measured. In some embodiments, peptides from the combination FOLH1, SPARC, TGFB1, CAMKK2, EGFR, and NCOA2 can be measured. For example, peptides from the combination FOLH1, SPARC, TGFB1, CAMKK2, and PSA can be measured.

In one example, the subject has previously had a prostatectomy, and the sample analyzed is a sample from the removed prostate (such as a formalin-fixed paraffin-embedded (FFPE) sample). In such an example, the methods of treating a subject with a risk of aggressive prostate cancer can include measuring or detecting the absolute or relative amounts of FOLH1, SPARC, TGFB1, CAMKK2 and PSA proteins in the prostatectomy sample, such as by measuring FOLH1, SPARC, TGFB1, CAMKK2 and PSA proteins or peptides thereof, antibodies, nucleic acid probes, or nucleic acid primers specific for FOLH1, SPARC, TGFB1, CAMKK2 and PSA. The expression levels of these molecules can be measured for example in the prostatectomy sample. If increased FOLH1, SPARC, TGFB1 and CAMKK2 protein or nucleic acid expression and decreased PSA protein or nucleic acid expression in the sample is measured or detected (for example as compared to a control representing expression for the aggressive prostate cancer-related molecule(s) expected in a sample from a subject who does not have aggressive prostate cancer), the methods can include administering therapeutic intervention to the subject, thereby treating the subject. In some examples the therapeutic intervention includes at least one of additional surgery (e.g., in addition to the prostatectomy), radiation, hormone therapy, chemotherapy, brachytherapy, cryotherapy, ultrasound, bisphosphate therapy, biologic therapy, or vaccine therapy, thereby treating the subject. In contrast, if expression of FOLH1, SPARC, TGFB1, and CAMKK2 is not increased, and PSA expression is not decreased in the prostatectomy sample (for example as compared to a control representing expression for the aggressive prostate cancer-related molecule(s) expected in a sample from a subject who does not have aggressive prostate cancer), the methods can include not administering therapeutic intervention to the subject. Instead, such a subject can receive monitoring, such as one or repeated biopsies or magnetic resonance imaging (MRI) at regular intervals or time points or watchful waiting (e.g., repeat biopsies and/or diagnostic imaging, such as MRI).

In one example, the subject has prostate cancer, but has not had a prostatectomy, and the sample analyzed is a sample from the prostate cancer (e.g., fine needle aspirate (FNA) or biopsy) or a blood sample. In such an example, the methods of treating a subject with a risk of aggressive prostate cancer can include measuring or detecting the absolute or relative amounts of FOLH1, SPARC, TGFB1, CAMKK2 and PSA proteins in the FNA, biopsy, or blood sample, such as by measuring FOLH1, SPARC, TGFB1, CAMKK2 and PSA proteins or peptides thereof, antibodies, nucleic acid probes, or nucleic acid primers specific for FOLH1, SPARC, TGFB1, CAMKK2 and PSA. The expression levels of these molecules can be measured. If increased FOLH1, SPARC, TGFB1 and CAMKK2 protein or nucleic acid expression and decreased PSA protein or nucleic acid expression in the sample is measured or detected (for example as compared to a control representing expression for the aggressive prostate cancer-related molecule(s) expected in a sample from a subject who does not have aggressive prostate cancer), the methods can include administering therapeutic intervention to the subject, thereby treating the subject. In some examples the therapeutic intervention includes administering a prostatectomy. In contrast, if expression of FOLH1, SPARC, TGFB1, and CAMKK2 is not increased, and PSA expression is not decreased in the prostatectomy sample (for example as compared to a control representing expression for the aggressive prostate cancer-related molecule(s) expected in a sample from a subject who does not have aggressive prostate cancer), the methods can include not subjecting the subject to a prostatectomy. Instead, such a subject can receive monitoring, such as such as one or repeated biopsies or magnetic resonance imaging (MRI) at regular intervals or time points or watchful waiting (e.g., repeat biopsies and/or diagnostic imaging, such as MRI).

In some examples, measuring expression of aggressive prostate cancer-related molecules, such as FOLH1, SPARC, TGFB1, CAMKK2, NCOA2, EGFR, or PSA, can include quantitating protein and/or nucleic acid expression of these molecules in a sample obtained from the subject. In particular examples, these molecules are first analyzed for measurement accuracy, such as correlating the amounts of different peptides from the same aggressive prostate cancer-related protein where the protein expression is measured.

In other examples, measuring increased protein or nucleic acid expression of FOLH1, SPARC, TGFB1, CAMKK2, NCOA2, or EGFR, or decreased expression of PSA is relative to an amount of FOLH1, SPARC, TGFB1, CAMKK2, NCOA2, EGFR, or PSA median protein or nucleic acid expression, respectively, for example a median value of protein or nucleic acid expression (such as using z-scoring) for each molecule expected in a subject without aggressive prostate cancer (e.g., as compared to a threshold of expression of any of these molecules established from a subject or subjects, such as a cohort of control subjects).

In some examples, measuring protein and/or nucleic acid expression of FOLH1, SPARC, TGFB1, CAMKK2, NCOA2, EGFR, or PSA can include measuring more than one molecules, such as 3, 4, 5, 6, or 7 of the molecules. In other examples, any combination of these molecules can be measured. In particular examples, the combination FOLH1, SPARC, and TGFB1; FOLH1, SPARC, TGFB1, and CAMKK; FOLH1, SPARC, TGFB1, CAMKK2, EGFR, and NCOA2; or FOLH1, SPARC, TGFB1, CAMKK2, and PSA can be measured.

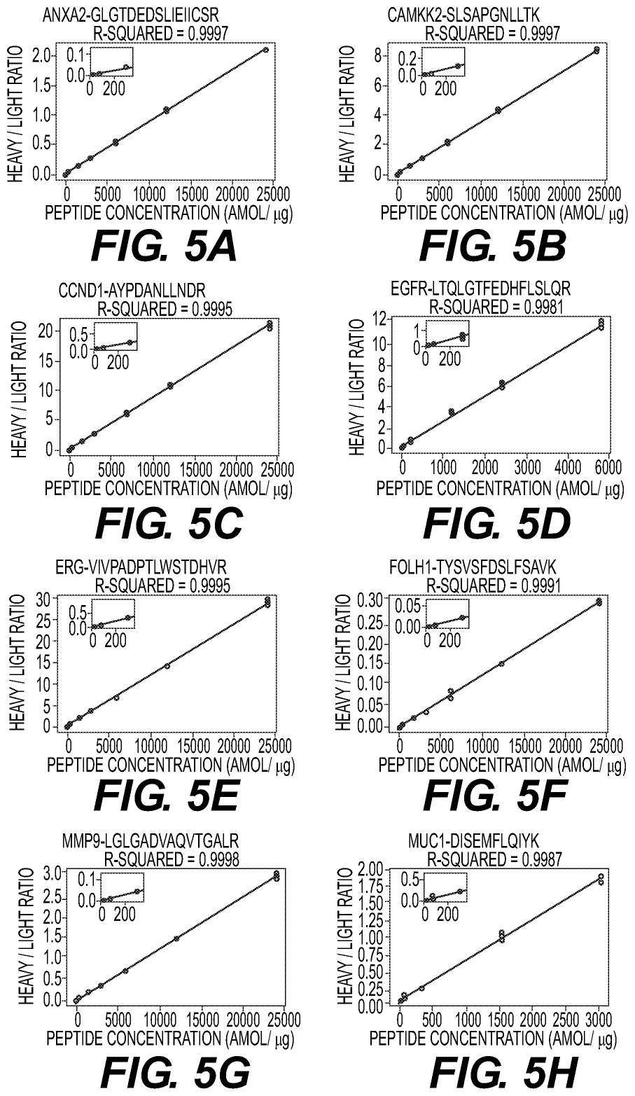

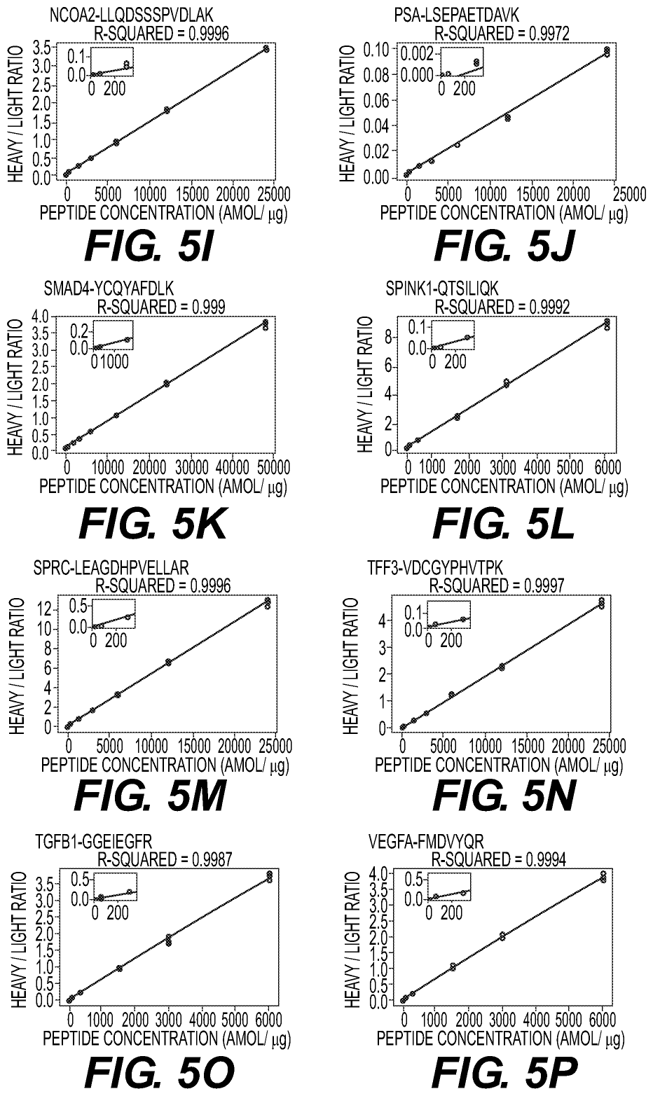

In some examples, measuring expression of aggressive prostate cancer-related molecules, such as FOLH1, SPARC, TGFB1, CAMKK2, NCOA2, EGFR, or PSA, can include measuring the amount of protein expressed. For example, measuring the amount of protein expressed can include measuring a peptide (e.g., see sequences provided in FIGS. 5A-5P) from the protein. More than one peptide can be measured for a protein, such as at least 2, at least 3, at least 4, at least 5, at least 6, at least 7, at least 8, at least 9, at least 10, at least 11, at least 12, at least 13, at least 14, at least 15, or at least 20 (such as 2, 3, 4, 5, 6, 7, 8, 9, 10, 11, 12, 13, 14, 15 or 20) peptides for a single protein. In some specific examples, peptides can be generated through contacting the protein (such as a sample containing the protein) with a protease, such a trypsin. Thus, in some examples, the methods include treating a sample to be analyzed with a protease, such as trypsin. In some particular examples, peptides for the aggressive prostate cancer-related molecules FOLH1, SPARC, TGFB1, CAMKK2, NCOA2, EGFR, or PSA include the peptides listed in FIGS. 5A-5P. In other particular examples, combinations of peptides for aggressive prostate cancer-related proteins can be used, such as the combinations FOLH1, SPARC, and TGFB1; FOLH1, SPARC, TGFB1, and CAMKK; FOLH1, SPARC, TGFB1, CAMKK2, EGFR, and NCOA2; and FOLH1, SPARC, TGFB1, CAMKK2, and PSA.

In embodiments, the subject with or at risk for aggressive prostate cancer includes a subject at risk for post-surgical biochemical recurrent prostate cancer (BCR). For example, for a subject at risk for BCR, the methods can include measuring the expression of the combination FOLH1, SPARC, TGFB1, CAMKK2, and PSA. In some examples where a subject is at risk for BCR, the methods can include measuring the combination FOLH1, SPARC, and TGFB1. In further examples, the methods can include measuring expression as compared to a control (such as a subject or subjects, for example, a cohort) representing expression for the aggressive prostate cancer-related molecule(s) expected in a sample from a subject who does not develop BCR (such as post-surgical BCR).

In embodiments, the subject with or at risk for aggressive prostate cancer includes a subject at risk for post-surgical distant metastatic prostate cancer (DM). For example, for a subject at risk for DM, the methods can include measuring the expression of the combination FOLH1, SPARC, TGFB1, CAMKK2, and PSA. In some examples where a subject is at risk for DM, the methods can include measuring the combination FOLH1, SPARC, TGFB1, and PSA. In further examples, the methods can include measuring expression as compared to a control (such as a subject or subjects, for example, a cohort) representing expression for the aggressive prostate cancer-related molecule(s) expected in a sample from a subject who does not develop DM (such as post-surgical DM).

In embodiments, the subject with or at risk for aggressive prostate cancer includes a subject at risk for high Gleason score (GS, such as about 7 (for example, the Gleason sum is 3+4 or 4+3), 8, 9, or 10, or about 7-10 or 8-10) or high Group Grade (GG, such as about 3, 4, or 5, or about 3-5 or 4-5). In some examples, where a subject is at risk for a high GS or GG, the methods can include measuring the combination FOLH1, SPARC, TGFB1, CAMKK2, EGFR, and NCOA2. In further examples, the methods can include measuring expression as compared to a control (such as a subject or subjects, for example, a cohort) representing expression for the aggressive prostate cancer-related molecule(s) expected in a sample from a subject who does not have a high GS or high GG.

In embodiments, the sample comprises a prostatectomy sample, a biopsy sample, blood sample, urine, semen, or expressed prostatic secretion sample. In specific, non-limiting examples, the sample is a prostatectomy sample, such as a formalin-fixed paraffin-embedded (FFPE) sample.

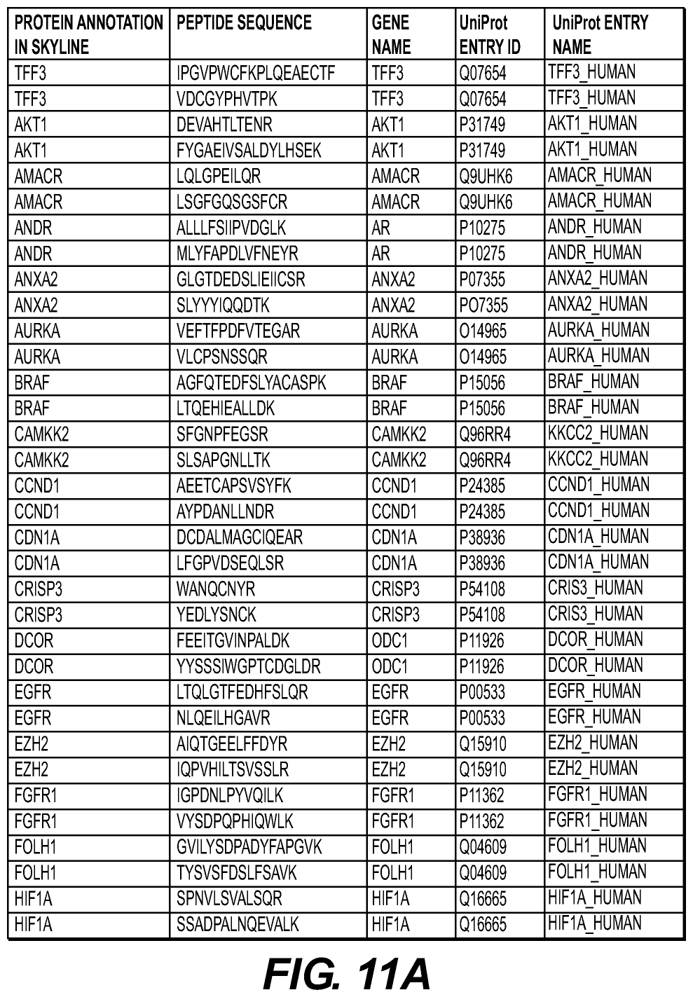

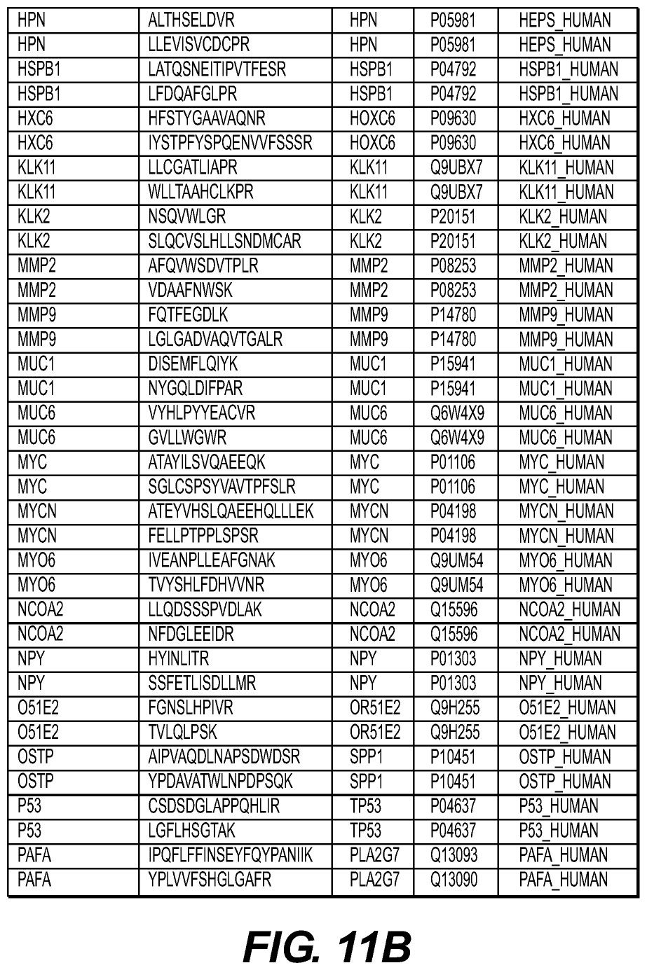

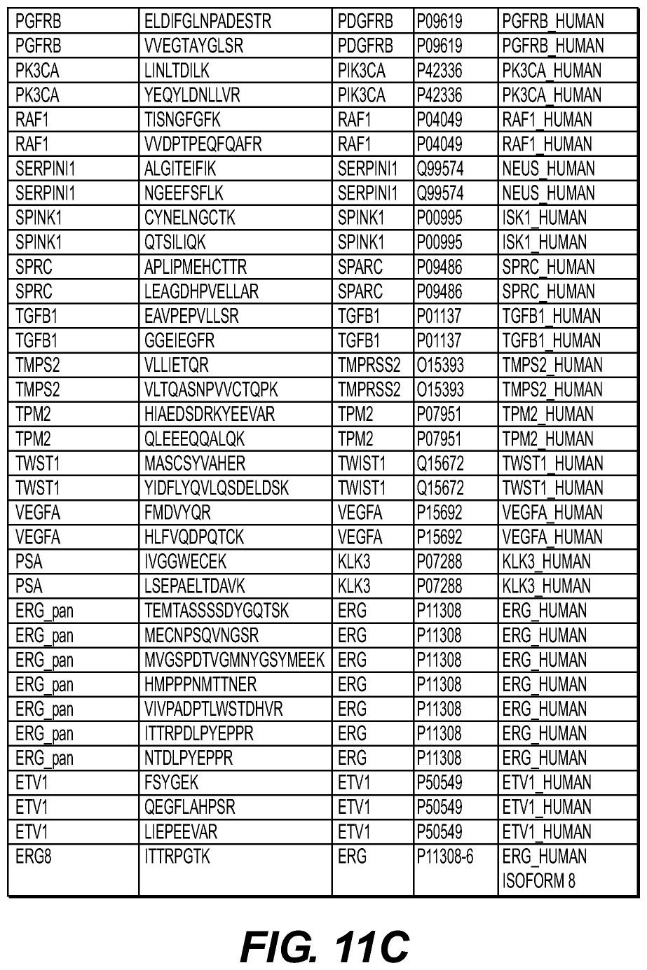

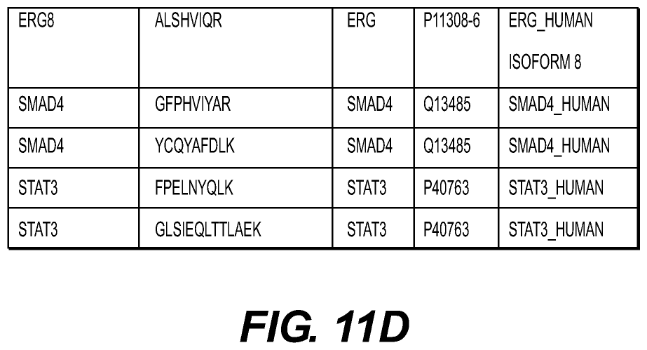

In embodiments, the expression is determined using relative protein or peptide abundance or concentration. For example, mass spectrometry can be used to determine the protein abundance or concentration of the full-length protein of peptide(s) thereof. In particular examples, mass spectrometry can be used to determine the protein abundance or concentration of aggressive prostate cancer-related molecules, such as FOLH1, SPARC, TGFB1, CAMKK2, NCOA2, EGFR, or PSA, using peptides, such as the peptides listed in FIGS. 5A-5P or FIGS. 11A-11D.

In particular examples, measuring expression of aggressive prostate cancer-related markers by using mass spectrometry can include using mass spectrometry assays such as LC-SRM, LG-SRM, and/or PRISM-SRM. In some examples, measuring expression of aggressive prostate cancer-related markers (such as in a prostatectomy sample) can include using an LC-SRM assay, for example, where the serum protein levels are least at a moderate abundance, such as about low μg/mL (e.g., 1-10, 10-50, 50-100, or 100-500 μg/mL).

In other examples, the measuring increased protein or nucleic acid expression of FOLH1, SPARC, TGFB1, CAMKK2, NCOA2, EGFR, or PSA includes measuring molecules that are at low-to-moderate abundance, for example, in the range of about low μg/mL to high ng/mL (e.g., 1-10 μg/mL, 500 ng/mL-1 μg/mL, or 100-500 ng/mL), in the sample obtained from the subject. In some examples, these molecules can be accurately measured by using assays with sufficient sensitivity, such as an LG-SRM assay or a PRISM-SRM assay.

In certain examples, measuring increased protein or nucleic acid expression of FOLH1, SPARC, TGFB1, CAMKK2, NCOA2, or EGFR, and optionally measuring decreased PSA protein expression, includes measuring some markers that are at low abundance (such as FOLH1, SPARC, TGFB1, CAMKK2, NCOA2, or EGFR), for example, in the range of about low ng/mL to high pg/mL (e.g., 500-100 ng/mL, 100-50 ng/mL, 50-10 ng/mL, 10-1 ng/mL, 500 pg/mL-1 ng/mL, 500-100 pg/mL, or 100-50 pg/mL) in the sample obtained from the subject. In some examples, these low-abundance markers can be accurately measured by using assays with sufficient sensitivity, such as a PRISM-SRM assay.

Other methods of determining expression of the aggressive prostate cancer-related molecules are possible, such as protein-, peptide-, or nucleic acid-based methods, as described herein. The methods herein can include a variety of additional steps, such as normalization.

In some examples, the methods can include measuring increased expression of two or more aggressive prostate cancer-related molecules, such as FOLH1, SPARC, TGFB1, CAMKK2, NCOA2, EGFR, or PSA, which can include the combinations FOLH1, SPARC, and TGFB1; FOLH1, SPARC, TGFB1, and CAMKK; FOLH1, SPARC, TGFB1, CAMKK2, EGFR, and NCOA2; or FOLH1, SPARC, TGFB1, CAMKK2, and PSA. In particular examples, the protein expression can be measured, for example, by using peptides of the proteins, such as the peptides in FIGS. 5A-5P. More than one peptide can be used for a protein, such as at least 2, at least 3, at least 4, at least 5, at least 6, at least 7, at least 8, at least 9, at least 10, at least 11, at least 12, at least 13, at least 14, at least 15, or at least 20 (such as 2, 3, 4, 5, 6, 7, 8, 9, 10, 11, 12, 13, 14, 15 or 20) peptides for a single protein. In specific examples, the amounts of peptides can be measured using mass spectrometry, such as LC-SRM, LG-SRM, and/or PRISM-SRM. In another example, the amounts of peptides are normalized. In specific examples, the increased expression measured for the aggressive prostate cancer-related molecules (such as 3, 4 5, 6, or 7 of FOLH1, SPARC, TGFB1, CAMKK2, NCOA2, EGFR, or PSA).

In specific embodiments, the methods disclosed herein can include treating a subject (such as a human subject) with aggressive prostate cancers by measuring expression of aggressive prostate cancer-related molecules in a sample obtained from a subject (such as a subject with or at risk of aggressive prostate cancer, for example, a subject with or at risk for BCR or DM). In examples, the aggressive prostate cancer-related molecules include FOLH1, SPARC, TGFB1, CAMKK2, and PSA. In examples, the methods further include measuring increased expression of the aggressive prostate cancer-related molecule(s) as compared to a control representing expression for the aggressive prostate cancer-related molecule(s) expected in a sample from a subject who does not have aggressive prostate cancer (such as a control subject that does not develop BCR or DM). In examples, the methods include administering at least one of surgery, radiation, hormone therapy, chemotherapy, brachytherapy, cryotherapy, ultrasound, bisphosphate therapy, biologic therapy, or vaccine therapy to the subject with aggressive prostate cancer, thereby treating the subject. In embodiments, the subject has had a prostatectomy, and the methods further include post-treatment surveillance.

### Evaluating Nucleic Acid Expression

In some examples, expression of nucleic acids (e.g., RNA, mRNA, cDNA, genomic DNA) of aggressive prostate cancer-related molecules, such as FOLH1, SPARC, TGFB1, CAMKK2, EGFR, NCOA2, or PSA, are analyzed and, in some examples, quantified. Suitable biological samples can include prostatectomy samples, biopsy samples, blood samples, urine, semen, expressed prostatic secretion samples, plasma samples, or serum samples obtained from a subject having or a subject at risk for aggressive prostate cancer (such as biochemical recurrent prostate cancer (BCR), distant metastasis (DM), or an intermediate to high Gleason score or Grade Group (GG)). An increase in the amount of nucleic acid molecules for the aggressive prostate cancer-related markers, such as A FOLH1, SPARC, TGFB1, CAMKK2, EGFR, and/or NCOA2, and optionally a decrease in PSA, in the sample indicates that the subject has aggressive prostate cancer, as described herein. In some examples, the assay is multiplexed, in that expression of several nucleic acids are detected simultaneously or contemporaneously (Quek et al., Prostate 75:1886-95, 2015). Other steps are possible, such as a normalization step.

Nucleic acid molecules can be isolated from a sample from a subject having or a subject at risk for aggressive prostate cancer, such as a prostatectomy sample, a biopsy sample, blood sample, urine, semen, expressed prostatic secretion sample, a plasma sample, or serum sample. In one example, RNA isolation is performed using a purification kit, buffer set, and protease from commercial manufacturers, such as QIAGEN®, according to the manufacturer's instructions. RNA prepared from a biological sample can be isolated, for example, by guanidinium thiocyanate-phenol-chloroform extraction, and oligp(dT)-cellulose chromatography (e.g., Tan et al., J Biomed Biotechnol., 2009: 574398, 10 pages, incorporated herein by reference in its entirety).

Methods of gene expression profiling include methods based on hybridization analysis of polynucleotides, methods based on sequencing of polynucleotides, and other methods in the art. In some examples, mRNA expression is quantified using northern blotting or in situ hybridization; RNase protection assays, or PCR-based methods, such as reverse transcription polymerase chain reaction (RT-PCR) or real time quantitative RT-PCR. Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE) and gene expression analysis by massively parallel signature sequencing (MPSS).

### Evaluating Protein Expression

In some examples, protein expression of aggressive prostate cancer-related molecules, such as FOLH1, SPARC, TGFB1, CAMKK2, EGFR, NCOA2, or PSA, is analyzed and, in some examples, quantified. Suitable biological samples include prostatectomy samples, biopsy samples, blood samples, urine, semen, expressed prostatic secretion samples, plasma samples, or serum samples obtained from a subject having or a subject at risk for aggressive prostate cancer, such as for BCR, DM, or an intermediate to high Gleason score or GG. An increase in the amount of aggressive prostate cancer-related marker proteins, such as FOLH1, SPARC, TGFB1, CAMKK2, EGFR, or NCOA2, proteins, and optionally a decrease in PSA, in the sample indicates that the subject has or is at risk for aggressive prostate cancer or BCR, DM, or a high GS or high GG, as described herein. In some examples, the assay is multiplexed, in that expression of several proteins is detected simultaneously or contemporaneously. Other steps are possible, such as a normalization step.

The expression of aggressive prostate cancer-related molecules, such as 2, 3, 4, 5, 6, or 7 of FOLH1, SPARC, TGFB1, CAMKK2, EGFR, NCOA2, or PSA, can be measured using any of a number of techniques, such as direct physical measurements (e.g., mass spectrometry) or binding assays (e.g., immunoassays, agglutination assays, and immunochromatographic assays, such as ELISA, Western blot, or RIA assay). Immunohistochemical techniques or immunohistochemistry (IHC) assay can also be utilized for protein detection and quantification.

In some embodiments, IHC assay may be done according to standard protocols, such as disclosed in Current Protocols in Molecular Biology (1991) 11.2.1-11.2.22. The proteins disclosed here can be detected by means of immunohistochemistry (IHC) on FFPE tissue samples using antibodies, or an antigen binding fragment thereof as primary antibody. For example, the FFPE tissue sample may be deparaffinized by placing dry paraffin sections on slides in a 60° C. oven for 1 hour. Subsequently, the slides may be placed in staining racks and immersed in staining dishes containing the following solutions: three times in xylene for 5 min each two times in 100% ethanol for at least 1 min each two times in 95% ethanol for at least 1 min each one time in 70% ethanol for at least 1 min. The slides may then be gently rinsed with tap water for about 5 minutes. Depending on the tissue the sample is derived from it may be required to block endogenous peroxidase activity by placing the slides in a 3% hydrogen peroxide solution for 10 minutes at room temperature, followed by a rinse with water. Subsequent antigen retrieval may be done in a water bath according to the following procedure: Slides may be placed in a Coplin jar with antigen retrieval solution such as Target Retrieval Solution, enhanced citrate buffer solution (Dako, S1699 or S1700), and Target Retrieval Solution, high pH (Dako, S3308), or 0.05M citrate buffer, pH 6, or Tris EDTA buffer, pH 8. Slides may then be allowed to equilibrate to 75° C. to 95° C. in a water bath and incubated for about 40 minutes. The slides may then be allowed to cool at room temperature for 20 min after which the solution is decanted and the slides may then be placed in a staining dish containing TBS/0.6% Tween 20 for a minimum of 5 minutes. Antigen retrieval may also be done using a PT link pretreatment module (DAKO) using Tris-EDTA buffer pH 9 at 97° C. for 20 minutes. Following the antigen retrieval the slides may then be subjected to the staining procedure using an automated instrument (e.g. Discovery XT., or AutostainerLink 48) following the manufacturer's instructions. For example, the slides may also be manually processed as described in Current Protocols in Molecular Biology 14.6.1-14.6.23, January 2008. For example, the slides may be covered with 400 to 500 μL of the antibody diluted into commercially available antibody diluent (e.g. from DAKO) to a concentration of about 0.2 μg/mL to about 10 μg/mL and incubated for about 30 minutes at room temperature in a moist chamber. The primary antibody may then be rinsed off with TBS/0.6% Tween-20. The slides may then be gently drained and freed from any remaining wash solution. Immediately thereafter the secondary antibody may be added and incubated at room temperature for about 30 min. Secondary antibody dilution may be from about 1:100 to about 1:10.000, from about 1:100, 1:150, 1:200, 1:250, 1:300, 1:400, 1:500, 1:750, 1:1000 to about 1:1500, 1:2000, 1:2500, 1:3000, 1:3500, 1:4000, 1:5000, 1:5500, 1:6000, 1:7000, 1:8000, 1:9000, or from about 1:100, 1:150, 1:200, 1:250, 1:300, 1:400, 1:500, 1:750 to about 1:1.000, 1:2000. Secondary antibodies that can be used for the detection of bound antibody may include HRP-conjugated immunoglobulins at a dilution of about 1:50, 1:175, to about 1:200, or goat anti-rabbit alkaline phosphatase (AP)-conjugated immunoglobulins at a dilution of about 1:20, 1:50, 1:100 to about 1:100, 1:200, 1:250, depending on the choice of the detection method and substrate employed, if Horseradish peroxidase (HRP)-conjugated secondary antibodies are used, 3,3′-diaminobenzidine (DAB) may be used for chromogenic detection, or 2,2′-azino-bis(3-ethylbenzothiazoline-6-sulphonic acid) (ABTS), or 3,3′,5,5′-Tetramethylbenzidine (TMB) may be used, or if AP-conjugated secondary antibodies are used, a substrate combination of nitro blue tetrazolium chloride (NBT) and 5-bromo-4-chloro-3-indolyl phosphate (BCIP) may be used. General principles and guidelines on chromogenic immunohistochemistry can be found in Current Protocols in Immunology 21.4.21-21.4.26, November 2013.

The method can include measuring or detecting a signal that results from a chemical reaction, such as a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques can detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). In some examples, detection techniques are used that do not require the use of labels, e.g., techniques based on measuring mass (e.g., surface acoustic wave measurements), refractive index (e.g., surface plasmon resonance measurements), or the inherent luminescence of an analyte, such as an aggressive prostate cancer-related molecule, for example, FOLH1, SPARC, TGFB1, CAMKK2, EGFR, NCOA2, or PSA.

For the purposes of quantitating proteins, a biological sample of the subject that includes cellular proteins (such as a prostatectomy sample) can be used. Quantitation of aggressive prostate cancer-related proteins, such as FOLH1, SPARC, TGFB1, CAMKK2, EGFR, NCOA2, or PSA proteins, can be achieved by immunoassay. The amount of aggressive prostate cancer-related proteins, such as FOLH1, SPARC, TGFB1, CAMKK2, EGFR, NCOA2, or PSA proteins, can be assessed in the sample, for example by contacting the sample with appropriate antibodies (or antibody fragments) specific for each protein, and then detecting a signal (for example present directly or indirectly on the antibody, for example by the use of a labeled secondary antibody).

In one example, an electrochemiluminescence immunoassay is used, such as the V-PLEX™ system (Meso Scale Diagnostics, Rockville, Md.). In such assays, the primary antibodies for aggressive prostate cancer-related proteins, such as FOLH1, SPARC, TGFB1, CAMKK2, EGFR, NCOA2, or PSA proteins, (or the corresponding secondary antibodies) are labeled with an electrochemiluminescent label.

Quantitative spectroscopic approaches methods, such as LC-SRM, LG-SRM, PRISM-SRM, and surface-enhanced laser desorption-ionization (SELDI), can be used to analyze expression of aggressive prostate cancer-related proteins, such as FOLH1, SPARC, TGFB1, CAMKK2, EGFR, NCOA2, or PSA proteins, in, for example, a prostatectomy sample obtained from a subject having or a subject at risk for aggressive prostate cancer, such as a subject having or at risk for BCR, DM, or high GS or GG. In some such spectroscopy methods, at least one peptide for each aggressive prostate cancer-related protein is measured or detected in the sample (e.g., FIGS. 5A-5P).

In one example, LC-SRM (liquid chromatography-selected reaction monitoring) may be used to detect protein expression for example by using a triple quadrupole spectrometer (see, e.g., U.S. Pub. No. 2013/0203096). LC-SRM is a liquid chromatography method that can be used for high-throughput selective and sensitive detection of molecules, such as aggressive prostate cancer-related proteins, for example, FOLH1, SPARC, TGFB1, CAMKK2, EGFR, NCOA2, or PSA. It can quantify moderately abundant analytes (low μg/mL) in limited sample volumes.

Therefore, in a particular example, the analytes can include aggressive prostate cancer-related proteins and/or peptides thereof, such as FOLH1, SPARC, TGFB1, CAMKK2, EGFR, NCOA2, or PSA proteins and/or peptides thereof. In other examples, the fractionated and pooled analytes consist essentially of or consist of FOLH1, SPARC, TGFB1, CAMKK2, EGFR, NCOA2, or PSA proteins or peptides thereof (such as in FIGS. 5A-5P), such as for the combinations of FOLH1, SPARC, and TGFB1; FOLH1, SPARC, TGFB1, and CAMKK; FOLH1, SPARC, TGFB1, CAMKK2, EGFR, and NCOA2; or FOLH1, SPARC, TGFB1, CAMKK2, and PSA. In this context, “consists essentially of” indicates that the fractionated and pooled analytes do not include other aggressive prostate cancer-related proteins that can be used to accurately predict aggressive prostate cancer, but can include other prostate molecules, such as prostate protein expression controls (such as for normalizing expression of protein or peptides thereof).

In another example, LG-SRM (long gradient-selected reaction monitoring) can be used to detect protein expression, for example by using a reversed-phase C18 column and triple quadrupole spectrometer (see, e.g., Shi et al., Anal Chem., 85 (19):9196-9203). LG-SRM is a liquid chromatography method for sensitive quantitation of analytes, such as aggressive prostate cancer-related proteins, and can even be used to accurately quantitate low-to-moderately abundant analytes (low μg/mL to high ng/mL).

In LG-SRM, a long, shallow LC gradient (e.g., 5 hours compared with a conventional LC protocol that can be about 45 min) using a long LC column is followed by SRM as a second step. The eluting LC peaks containing the target analyte, such as aggressive prostate cancer-related proteins or surrogate peptides thereof, for example, FOLH1, SPARC, TGFB1, CAMKK2, EGFR, NCOA2, or PSA proteins or peptides thereof, are, thus, sufficiently separated and resolved for accurate quantitation via SRM.

Therefore, in a particular example, the target analytes include aggressive prostate cancer-related marker proteins and/or surrogate peptides thereof, such as FOLH1, SPARC, TGFB1, CAMKK2, EGFR, NCOA2, or PSA proteins and/or peptides thereof. In other examples, the target analytes consist essentially of or consist of FOLH1, SPARC, TGFB1, CAMKK2, EGFR, NCOA2, or PSA proteins or peptides thereof; of the combinations of proteins or peptides listed in FIGS. 5A-5P. In this context “consists essentially of” indicates that the target analytes do not include other aggressive prostate cancer-related proteins that can be used to accurately predict aggressive prostate cancer, but can include other prostate molecules, such as prostate protein expression controls (such as for normalizing expression of proteins or peptides thereof).

In an additional example, PRISM-SRM (high-pressure, high-resolution separations, intelligent selection, multiplexing-selected reaction monitoring) is used to detect protein expression, for example, by using an ultra-pressure LC (UPLC) system and a triple quadrupole spectrometer (see, e.g., U.S. Pub. No. 2014/0194304; Shi et al., PNAS, 109 (38):15395-15400 (2012); and Shi et al., J Proteome Res., 13 (2):875-882 (2014)). PRISM-SRM is a liquid chromatography method for quantitating analytes, such as aggressive prostate cancer-related proteins, and can even be used to accurately quantitate low-abundance (low ng/mL to high pg/mL) analytes.

In PRISM-SRM, LC-SRM is used as a second step after the target analyte is enriched through a liquid chromatography pre-fractionation step, such as using reverse-phase chromatography. The fractions containing the target analyte, such as aggressive prostate cancer-related proteins or peptides thereof, for example, FOLH1, SPARC, TGFB1, CAMKK2, EGFR, NCOA2, or PSA proteins or peptides thereof, can then be pooled. The pooled fractions are enriched in the target analyte(s) and can then undergo a second LC separation step followed by SRM analysis.

Therefore, in a particular example, the fractionated and pooled analytes include aggressive prostate cancer-related proteins or peptides thereof, such as FOLH1, SPARC, TGFB1, CAMKK2, EGFR, NCOA2, or PSA proteins or peptides thereof. In other examples, the fractionated and pooled analytes consist essentially of or consist of FOLH1, SPARC, TGFB1, CAMKK2, EGFR, NCOA2, or PSA proteins or peptides thereof; of the combinations of proteins or peptides listed in FIGS. 5A-5P. In this context “consists essentially of” indicates that the fractionated and pooled analytes do not include other aggressive prostate cancer-related proteins that can be used to accurately predict aggressive prostate cancer, but can include other prostate molecules, such as prostate protein expression controls (such as for normalizing expression of proteins or peptides thereof).

In a further example, surface-enhanced laser desorption-ionization time-of-flight (SELDI-TOF) mass spectrometry is used to detect protein expression, for example, by using the ProteinChip™ (Ciphergen Biosystems, Palo Alto, Calif.).

### Aggressive Prostate Cancer-Related Molecules

The disclosed aggressive prostate cancer-related molecules include FOLH1, SPARC, TGFB1, CAMKK2, EGFR, NCOA2, or PSA. Three or more of the disclosed aggressive prostate cancer-related molecules can be used alone or in any combination. The molecules can include proteins, peptides (e.g., peptides, see FIGS. 5A-5P for example), and nucleic acids.

In some embodiments, one of the disclosed aggressive prostate cancer-related molecules can include FOLH1 (e.g., SEQ ID NO: 1). In some embodiments, one of the disclosed aggressive prostate cancer-related molecules can include SPARC (e.g., SEQ ID NO: 2). In some embodiments, one of the disclosed aggressive prostate cancer-related molecules can include TGFB1 (e.g., SEQ ID NO: 3). In some embodiments, one of the disclosed aggressive prostate cancer-related molecules can include PSA (e.g., SEQ ID NO: 4). In some embodiments, one of the disclosed aggressive prostate cancer-related molecules can include CAMKK2 (e.g., SEQ ID NO: 5). In some embodiments, one of the disclosed aggressive prostate cancer-related molecules can include EGFR (e.g., SEQ ID NO: 6). In some embodiments, one of the disclosed aggressive prostate cancer-related molecules can include NCOA2 (e.g., SEQ ID NO: 7). In some examples, combinations of these aggressive prostate cancer-related molecules are used, such as 3, 4, 5, 6, or 7 of these.

Molecules that are similar to the aggressive prostate cancer-related molecules disclosed above can be used as well as fragments thereof that retain biological activity. These molecules may contain variations, substitutions, deletions, or additions (e.g., the variation carbamidomethyl cysteine may be used instead of cysteine). The differences can be in regions not significantly conserved among different species. Such regions can be identified by aligning the amino acid sequences of related proteins from various animal species. Generally, the biological effects of a molecule are retained. For example, a molecule at least 75%, 80%, 85%, 90%, 95%, 96%, 97%, 98% or 99% identical to one of these molecules can be utilized. Molecules are of use that include at most 1, 2, 3, 4, 5, 6, 7, 8, 9, or 10 conservative amino acid substitutions. Generally, molecules are of use provided they retain at least 75%, 80%, 85%, 90%, 95%, 96%, 97%, 98% or 99% of the biological function of the native molecule, or have increased biological function as compared to the native molecule.

### Administration of Therapy

Subjects analyzed with the disclosed methods and who are found to have aggressive prostate cancer or at risk of developing aggressive prostate cancer can be selected for treatment. For example, subjects with aggressive prostate cancer or at risk of developing aggressive prostate cancer (such as a subject with BCR, DM, or a high GS or GG) found to have increased expression of FOLH1, SPARC, TGFB1, CAMKK2, EGFR, NCOA2, or PSA can be administered therapy for aggressive prostate cancer. Currently, the standard of care for prostate cancer can vary, but aggressiveness can be a factor. For example, a subject may be found to have aggressive prostate cancer, such as a patient with increased levels of FOLH1, SPARC, TGFB1, CAMKK2, EGFR, and/or NCOA2, and decreased levels of PSA. In some examples, subjects without aggressive prostate cancer may be treated using watchful waiting or active surveillance, both of which entail monitoring the cancer for changes and the subject for symptoms. Given that more invasive treatments entail a greater potential for side effects, active surveillance can be used for patients without aggressive prostate cancer.

In other examples, surgical removal of the prostate can be a treatment for aggressive prostate cancer or prostate cancers that do not respond to radiation therapy. In additional examples, subjects with aggressive prostate cancer, such as subjects with increased expression of FOLH1, SPARC, TGFB1, CAMKK2, EGFR, and/or NCOA2, and decreased levels of PSA can be treated with radiation therapy, such as using ionizing radiation to kill prostate cancer cells. In some other examples, subjects with aggressive prostate cancer or at risk of developing aggressive prostate cancer can be treated using brachytherapy, for example, where small radioactive particles, such as iodine-125 or palladium-103, are directly injected into the tumor, providing localized X-rays at the site of the tumor. In additional examples, ultrasound, such as high-intensity focused ultrasound (HIFU) is used where a subject has a recurrent case of prostate cancer (or BCR), such as where a subject was successfully treated for prostate cancer but subsequently had increased expression of FOLH1, SPARC, TGFB1, CAMKK2, EGFR, NCOA2, or PSA.

In further examples, a subject can be treated with hormone therapy, such as by modulating the levels of testosterone in the body, where the subject has or is at risk for without aggressive prostate cancer, for example, a subject that was successfully treated for prostate cancer but subsequently had increased expression of FOLH1, SPARC, TGFB1, CAMKK2, EGFR, NCOA2, or PSA, compared with the expression expected from a patient without aggressive prostate cancer (e.g., as compared to a threshold of expression of any of these molecules established from one or more subjects, such as a cohort of control subjects).

In some examples, at least a portion of the prostate cancer is surgically removed (for example via cryotherapy), irradiated, chemically treated (for example via chemoembolization), or combinations thereof, as part of the therapy. For example, a subject having aggressive prostate cancer can have all or part of the tumor surgically excised prior to administration of additional therapy.

Exemplary agents that can be used include one or more anti-neoplastic agents, such as radiation therapy, chemotherapeutic, biologic (e.g., immunotherapy), and anti-angiogenic agents or therapies. Methods and therapeutic dosages of such agents are known to those skilled in the art, and can be determined by a skilled clinician. These therapeutic agents (which are administered in therapeutically effective amounts) and treatments can be used alone or in combination. In some examples, 1, 2, 3, 4 or 5 different anti-neoplastic agents are used as part of the therapy.

In one example the therapy includes administration of one or more chemotherapy immunosuppressants (such as Rituximab, steroids) or cytokines (such as GM-CSF). Chemotherapeutic agents are known (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Exemplary chemotherapeutic agents that can be used with the therapy include but are not limited to, carboplatin, cisplatin, paclitaxel, docetaxel, doxorubicin, epirubicin, cabaziatxel, estramustine, vinblastine, topotecan, irinotecan, gemcitabine, iazofurine, etoposide, vinorelbine, tamoxifen, valspodar, cyclophosphamide, methotrexate, fluorouracil, mitoxantrone, and Doxil® (liposome encapsulated doxiorubicine). In one example, the therapy includes docetaxel and prednisone. In one example, the therapy includes cabaziatxel.

In one example, the therapy includes administering one or more of a microtubule binding agent, DNA intercalator or cross-linker, DNA synthesis inhibitor, DNA and/or RNA transcription inhibitor, antibodies, enzymes, enzyme inhibitors, and gene regulators.

Microtubule-binding agents interact with tubulin to stabilize or destabilize microtubule formation thereby inhibiting cell division. Examples of microtubule binding agents that can be used as part of the therapy include, without limitation, paclitaxel, docetaxel, vinblastine, vindesine, vinorelbine (navelbine), the epothilones, colchicine, dolastatin 10, nocodazole, and rhizoxin. Analogs and derivatives of such compounds also can be used. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds can be used as part of the therapy: suitable DNA and/or RNA transcription regulators, including, without limitation, anthracycline family members (for example, daunorubicin, doxorubicin, epirubicin, idarubicin, mitoxantrone, and valrubicin) and actinomycin D, as well as derivatives and analogs thereof. DNA intercalators and cross-linking agents that can be administered to a subject include, without limitation, platinum compounds (for example, carboplatin, cisplatin, oxaliplatin, and BBR3464), mitomycins, such as mitomycin C, bleomycin, chlorambucil, cyclophosphamide, as well as busulfan, dacarbazine, estramustine, and temozolomide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, exemestane, trichostatin and derivatives and analogs thereof Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone, and derivatives and analogs thereof. Kinase inhibitors include imatinib, gefitinib, and erolitinib that prevent phosphorylation and activation of growth factors.

In one example, the therapy includes folic acid (for example, methotrexate and pemetrexed), purine (for example, cladribine, clofarabine, and fludarabine), pyrimidine (for example, capecitabine), cytarabine, fluorouracil, gemcitabine, and derivatives and analogs thereof. In one example, the therapy includes a plant alkaloid, such as podophyllum (for example, etoposide) and derivatives and analogs thereof. In one example, the therapy includes an antimetabolite, such as cytotoxic/antitumor antibiotics, bleomycin, hydroxyurea, mitomycin, and derivatives and analogs thereof. In one example, the therapy includes a topoisomerase inhibitor, such as a topoisomerase I inhibitor (e.g., topotecan, irinotecan, indotecan, indimitecan, camptothecin and lamellarin D) or a topoisomerase II inhibitor (e.g., etoposide, doxorubicin, daunorubicin, mitoxantrone, amsacrine, ellipticines, aurintricarboxylic acid, ICRF-193, genistein, and HU-331), and derivatives and analogs thereof. In one example, the therapy includes a photosensitizer, such as aminolevulinic acid, methyl aminolevulinate, porfimer sodium, verteporfin, and derivatives and analogs thereof. In one example, the therapy includes a nitrogen mustard (for example, chlorambucil, estramustine, cyclophosphamide, ifosfamide, and melphalan) or nitrosourea (for example, carmustine, lomustine, and streptozocin), and derivatives and analogs thereof.

Other therapeutic agents, for example anti-tumor agents, that may or may not fall under one or more of the classifications above, also are suitable for therapy. By way of example, such agents include adriamycin, apigenin, rapamycin, zebularine, cimetidine, amsacrine, anagrelide, arsenic trioxide, axitinib, bexarotene, bevacizumab, bortezomib, celecoxib, estramustine, hydroxycarbamide, lapatinib, pazopanib, masoprocol, mitotane, tamoxifen, sorafenib, sunitinib, vandetanib, tretinoin, and derivatives and analogs thereof.

In one example, the therapy includes one or more biologics, such as a therapeutic antibody, such as monoclonal antibodies. Examples of such biologics that can be used include one or more of bevacizumab, cetuximab, panitumumab, pertuzumab, trastuzumab, bevacizumab (Avastin®), ramucirumab, and the like. In specific examples, the antibody or small molecules used as part of the therapy include one or more of the monoclonal antibodies cetuximab, panitumumab, pertuzumab, trastuzumab, bevacizumab (Avastin®), ramucirumab, or a small molecule inhibitor such as gefitinib, erlotinib, and lapatinib.

In some examples, the therapy includes administration of one or more immunotherapies, which may include the biologics listed herein. In specific examples, the immunotherapy includes therapeutic cancer vaccines, such as those that target PSA (e.g., ADXS31-142), prostatic acid phosphatase (PAP) antigen, TARP, telomerase (e.g., GX301) or that deliver 5T4 (e.g., ChAdOx1 and MVA); antigens NY-ESO-1 and MUC1; antigens hTERT and survivin; prostate-specific antigen (PSA) and costimulatory molecules (e.g., LFA-3, ICAM-1, and B7.1) directly to cancer cells, such as rilimogene galvacirepvac. Other examples of therapeutic vaccines include DCVAC, sipuleucel-T, pTVG-HP DNA vaccine, pTVG-HP, JNJ-64041809, PF-06755992, PF-06755990, and pTVG-AR. In other examples, the immunotherapy includes oncolytic virus therapy, such as aglatimagene besadenovec, HSV-tk, and valacyclovir. In additional examples, the immunotherapy can include checkpoint inhibitors, such as those that target PD-1 (e.g., nivolumab, pembrolizumab, durvalumab, and atezolizumab), CTLA-4 (e.g., tremelimumab and ipilimumab), B7-H3 (e.g., MGA271), and CD27 (e.g., CDX-1127). The protein MGD009 may also be used in another example. In specific examples, the immunotherapy can also include adoptive cell therapy, such as those that include T cells engineered to target NY-ESO-1 and those that include natural killer (NK) cells. In some examples, the immunotherapy can include adjuvant immunotherapies, such as sipuleucel-T, indoximod, and mobilan. In other specific examples, the immunotherapy includes one or more of tisotumab vedotin, sacituzumab govitecan, LY3022855, BI 836845, vandortuzumab vedotin, and BAY2010112, and MOR209/ES414. In additional examples, the immunotherapy can include cytokines, such as CYT107, AM0010, and IL-12.

In some examples, the subject receiving the therapy is also administered interleukin-2 (IL-2), as part of the therapy, for example via intravenous administration. In particular examples, IL-2 is administered at a dose of at least 500,000 IU/kg as an intravenous bolus over a 15-minute period every eight hours beginning on the day after administration of the peptides and continuing for up to 5 days. Doses can be skipped depending on subject tolerance.

In some examples, the subject receiving the therapy is also administered a fully human antibody to cytotoxic T-lymphocyte antigen-4 (anti—CTLA-4) as part of the therapy, for example via intravenous administration. In some example subjects receive at least 1 mg/kg anti-CTLA-4 (such as 3 mg/kg every 3 weeks or 3 mg/kg as the initial dose with subsequent doses reduced to 1 mg/kg every 3 weeks).

In one specific example for a subject with aggressive prostate cancer, such as a subject with increased expression of FOLH1, SPARC, TGFB1, CAMKK2, EGFR, NCOA2, or PSA, the therapy can include one or more of abiraterone acetate, bicalutamide, cabazitaxel, casodex (bicalutamide), degarelix, docetaxel, enzalutamide, flutamide, goserelin acetate, jevtana (cabazitaxel), leuprolide acetate, lupron (leuprolide acetate), lupron depot (leuprolide acetate), lupron depot-3 month (leuprolide acetate), lupron depot-4 month (leuprolide acetate), lupron depot-ped (leuprolide acetate), mitoxantrone hydrochloride, nilandron (nilutamide), nilutamide, provenge (sipuleucel-t), radium 223 dichloride, sipuleucel-T, taxotere (docetaxel), viadur (leuprolide acetate), xofigo (radium 223 dichloride), xtandi (enzalutamide), zoladex (goserelin acetate), and zytiga (abiraterone acetate).

In another specific example for a subject with aggressive prostate cancer, such as a subject with increased expression of FOLH1, SPARC, TGFB1, CAMKK2, EGFR, and/or NCOA2, and decreased levels of PSA, the therapy can include one or more of chemotherapy drugs, such as cabazataxel (Jevtana®), docetaxel (Taxotere®), mitoxantrone (Teva®), or androgen deprivation therapy (ADT), such as with abiraterone Acetate (Zytiga®), bicalutamide (Casodex®), buserelin Acetate (Suprefact®), cyproterone Acetate (Androcur®), degarelix Acetate (Firmagon®), enzalutamide (Xtandi®), flutamide (Euflex®), goserelin Acetate (Zoladex®), histrelin Acetate (Vantas®), leuprolide Acetate (Lupron®, Eligard®), triptorelin Pamoate (Trelstar®). The therapy can also include drugs to treat bone metastases (bisphosphate therapy), such as alendronate (Fosamax®), denosumab (Xgeva®), pamidronate (Aredia®), zoledronic acid (Zometa®), or radiopharmaceuticals, such as radium 223 (Xofigo®), strontium-89 (Metastron®), and samarium-153 (Quadramet®).

The therapy can be administered in cycles (such as 1 to 6 cycles), with a period of treatment (usually 1 to 3 days) followed by a rest period. But some therapies can be administered every day.

### EXAMPLES

Improved clinical management of prostate cancer is based on early detection of neoplastic lesions in the prostate and early discrimination of indolent prostate cancer, which can be effectively managed by active surveillance, from aggressive forms of prostate cancer, which can rapidly metastasize and develop castration resistance. An initial panel of candidate biomarkers was selected and then filtered based on differential expression using mRNA and NanoString. Targeted proteomic assays were developed for 52 differentially expressed genes and examined for detecting cognate proteins in FFPE-preserved RP specimens from a small patient cohort with long-term follow-up, which documented distant metastasis, BCR, and no progression events. Of the 42 proteins that were detected and quantified, 16 were validated in a subset of 105 patients and then further examined in a combined cohort of 338 patients.

A resulting panel of three target proteins were robustly associated with BCR, four with distant metastasis, and six with high GG; across these proteins, 3 were in common for all endpoints: FOLH1, SPARC, and TGFB1. The nested experimental design used herein provided stringent metrics for reproducibility and robustness, and the final cohort of 338 patients is one of the largest tested for validation of such proteomic biomarkers.

Tissue proteomic classifiers disclosed herein significantly improved performance of the biopsy base model for predicting either BCR or metastasis; a similar trend was demonstrated when the three-protein panel was combined with a pathology base model, showing statistically significant improvement in detecting metastasis. With AUC values of 0.92 for predicting metastasis and over 0.72 for predicting BCR, this proteomic classifier exhibits clinical utility.

### Example 1

### Materials and Methods

Demographic, clinical, and treatment variables. Age at PCa diagnosis (years), self-reported race (African American, Caucasian American, and “Other”), PSA at PCa diagnosis (ng/mL), clinical T stage (T1-T2a, T2b-T2c, T3a-T4), biopsy Gleason sum (≤6, 7, 8-10), NCCN-defined risk strata (low, intermediate risk, and high risk), time from diagnosis to RP (months), and post-RP follow-up time (months) were considered.

RP specimen processing and pathologic variable measurement. All RP specimens were processed by whole mount and sectioned at 2.2-mm intervals. Pathologic parameters were measured based on evaluation by central pathology review, including pathologic T stage (pT2, pT3-pT4), grade group (GG1-5), and surgical margin status (negative, positive).

Analysis of FFPE tissue samples using PRISM-SRM. The protocols for PRISM-SRM analysis of the FFPE prostatectomy tissue samples included deparaffinization, protein extraction, reduction, alkylation, tryptic digestion, and desalting, which was followed by PRISM fractionation and LC-SRM analysis of the resulting peptide fractions. FFPE samples for the entire cohort were randomized and analyzed by PRISM-SRM at PNNL in a blinded fashion (patient outcome data were restricted at CPDR during the entire analysis from the experimental design to statistical analysis).

Development of PRISM-SRM assays. High-sensitivity PRISM-SRM assays for the candidate proteins were developed using synthetic peptides.

Protein quantification from the PRISM-SRM data. The raw data acquired on the TSQ Vantage triple quadrupole MS were imported into Skyline software for visualization and quantification. The total peak area ratio between endogenous peptides and their heavy isotope labeled peptide standards was used for quantification. Peak detection and integration were based on two criteria: 1) the same retention time and 2) approximately the same relative peak intensity ratios across multiple transitions between endogenous peptide and heavy isotope labeled peptide standards. All data were manually inspected to ensure correct peak detection and accurate integration. The concentration of proteins was calculated by the measured light over heavy peak area ratio and the response curve.

Dependent study outcomes. To ascertain whether targeted protein marker expression in FFPE tissues could be used to predict PCa progression, the study outcomes included BCR and distant metastasis after RP. A BCR event was defined in the following manner: a post-RP PSA level >0.2 ng/mL followed by a successive, confirmatory PSA level ≥0.2 or the initiation of salvage radiation or hormonal therapy after a rising PSA, excluding PSA values drawn within eight weeks of the RP.

Presence of distant metastasis was ascertained by physician's review of each patient's complete imaging history, including bone scan, CT scan, MRI, and/or bone biopsy results. Subjects who had no evidence of BCR or metastasis at the end of study period with at least 10 years of post-RP follow-up were defined as “non-events”.

Statistical analysis. Analysis of variances (ANOVA) and Kruskal-Wallis test were used to examine the differences of distribution of continuous variables across event groups (non-event, BCR, and metastasis), while Chi-square testing or Fisher exact tests were used to evaluate the associations of categorical clinical-pathological variables across event groups. Unadjusted Kaplan-Meier survival analysis and log-rank testing were used to examine the DM-free survival curves stratified by the protein classifier cut point. Whisker boxplots were used to show differences in protein marker distribution across the 3 event groups. Univariable logistic regression and ROC curve analysis were used to identify which protein markers were significantly associated with the three event groups, and the significance level was adjusted using the Bonferroni method. Using multivariable logistic regression analysis, ROC curves were generated to evaluate prediction accuracy based on AUC statistics of each set of protein marker “panels” for each outcome. For each ROC curve analysis, the protein panel was examined in combination with SOC variables versus the SOC variables alone. Two “base” models comprised of SOC variables were examined: 1) a biopsy “base” model, including patient age at PCa diagnosis, self-reported race, and NCCN risk stratum and 2) a pathology “base” model, including: age at PCa diagnosis, self-reported race, pathological T stage, GG, and surgical margin status. Multivariable Cox proportional hazards (PH) models were constructed to examine distant metastasis-free survival and BCR-free survival as a function of both the protein marker panels specific to each study endpoint (metastasis, BCR), adjusting for the biopsy “base” model covariates and followed by adjustment for the pathologic “base” model covariates.

All 95% confidence intervals (CI) for AUC values were constructed using a non-parametric, bootstrapping method with 1,000 replicates. All statistical analysis was performed using SAS version 9.4 (North Carolina) and statistical significance was set at p<0.05 (except for univariable analysis of individual protein markers, described previously).

Protein digestion of FFPE tissue samples. The FFPE human prostate tissue samples were first deparaffinized by adding 500 μL of xylene (Sigma Aldrich, St. Louis, Mo.) and incubating for 5 min at room temperature with a 300 rpm shaker speed. The solution was removed, the xylene added, and incubation repeated. After removing the solution a second time, 500 μL of 190-proof ethanol (Decon Laboratories, King of Prussia, Pa.) was added, and the sample was incubated for 5 min at room temperature with a 300 rpm shaker speed. The solution was removed. Finally, 500 μL of 80% ethanol was added and incubated for 5 min at room temperature with a 300 rpm shaker speed. The solution was removed, and the samples were dried for 15 min in a Speed-Vac (Thermo Savant).

Once dried, 50 μL of 2,2,2-trifluoroethanol (TFE) (Sigma Aldrich) was added to the samples. Next, 50 μL of 600 mM Tris-HCl was added to the samples for a final concentration of 50% TFE. The samples were homogenized with a Kontes® Pellet Pestle® for 30 seconds, keeping the samples cool on an ice block during homogenization and afterwards for 3 min. The samples were transferred to a 1.5-mL screw top tube before incubating with a Thermomixer (Eppendorf, Hamburg, Germany) at 99° C. for 90 min with a 1000 rpm shaker speed. The samples were allowed to cool to room temperature. The protein concentration of the samples was determined using BCA assay (Thermo Fisher Scientific, Waltham, Mass.).

Proteins were reduced with 5 mM Dithiothreitol at 37° C. for 1 hr and alkylated using 10 mM iodoacetamide at room temperature for 1 hr in the dark. The samples were diluted with water and digested with sequencing grade modified trypsin (Promega Corporation, Madison, Wis.) at a 1:50 trypsin:protein ratio. The samples were incubated at 37° C. for 4 hr, then 1:50 trypsin was added a second time, and the samples were incubated overnight at 37° C. The digestion was stopped by adding 10% formic acid to reach a final concentration of 1% formic acid.

The samples were centrifuged at 14,000 rpm at 4° C. prior to the final solid-phase extraction (SPE) based desalting step using 50 mg, 1 mL C-18 SPE cartridges (Strata, Phenomenex, Torrance, Calif.) and a manual vacuum manifold (Supelco, Sigma Aldrich). The cartridges were preconditioned using 3 mL of 100% methanol followed by 2 mL of 0.1% TFA. The sample was loaded and slowly passed through the cartridge at a rate no faster than 1 mL per minute. The cartridge was then washed with 4 mL of 5% ACN and 0.1% TFA and with 1 mL of 1% formic acid to remove any residual TFA. The desalted peptide sample was eluted into a 2.0-mL microcentrifuge tube using 1.5 mL of 80% ACN and 0.1% formic acid. The eluted sample was placed in the Speed-Vac and concentrated. The peptide concentration was determined using the BCA assay, and the final concentration was adjusted to 0.3 μg/μL. The sample was then frozen in liquid nitrogen and stored at −70° C. until needed for peptide spiking and SRM analysis.

Heavy isotope-labeled peptides and heavy peptides mixture stock. A total of 110 tryptic peptides for the 52 protein candidates were selected based on well-accepted criteria for targeted proteomics analysis [1]. Pure stable isotope-labeled heavy peptides (purity>97%) with C-terminal [U-13C6, 15N2] lysine or [U-13C6, 15N4] arginine were synthesized (AQUA QuantPro, ThermoFisher Scientific, Waltham, Mass.) for PRISM-SRM assay development and measurements. The peptide list is provided in FIGS. 11A-11D. The peptides were received at a concentration of 5 pmol/μL in 5% ACN. Equal volume of these 110 peptides were mixed together to create a heavy peptide mixture stock, and the final peptide concentration in the stock was 45 fmol/μL.

SRM transitions and collision energy. The transitions and collision energy of individual peptides were first optimized by direct infusion experiments on a TSQ Vantage triple quadrupole mass spectrometer (ThermoFisher Scientific, Waltham, Mass.) and furthered evaluated by LC-SRM using a nanoACQUITY UPLC® system (Waters Corporation, Milford, Mass.) and a TSQ Vantage triple quadrupole mass spectrometer. The three best transitions with minimal interference and highest sensitivity were retained for each peptide in the final SRM assays.

PRISM-SRM assay configuration. Heavy peptides were spiked in digested and cleaned FFPE samples, and they were separated following the PRISM workflow using high pH reversed-phase capillary LC on a nanoACQUITY UPLC® system as described previously [2]. Briefly, separations were performed using a capillary column packed in-house (3 μm Jupiter C18 bonded particles, 200 μm i.d.×50 cm long) at a flow rate of 2.2 μL/min on binary pump systems using 10 mM ammonium formate (pH 9) as mobile phase A and 10 mM ammonium formate in 90% ACN (pH 9) as mobile phase B. Forty-five μL of each sample (35 μg) were loaded onto the column and separated using a binary gradient of 5-15% B in 15 min, 15-25% B in 25 min, 25-45% B in 25 min, and 45-90% B in 38 min. The samples were separated into 96 fractions (1-min elution time per fraction), and the fractions were collected using a LEEP's collect PAL (LEAP Technologies, Carrboro, N.C.). Prior to peptide fraction collection, ˜20 μL of water was added to each well in the plate to avoid peptide loss and to dilute the peptide fraction for LC-SRM analysis.

Configuration 1: 110 peptides (52 proteins) in the first 105 samples. To facilitate the high-throughput PRISM-SRM analysis of 110 peptides in the first batch of 105 samples, the 96 fractions were concatenated into 24 fractions. These 24 fractions were analyzed individually on the second dimension LC-SRM using a nanoACQUITY UPLC® system coupled to TSQ Vantage triple quadrupole mass spectrometer. Briefly, separations were performed using a ACQUITY UPLC M-Class Peptide BEH C18 Column, 300 Å, 1.7 μm, 100 μm×100 mm (Waters Cooperation) at a flow rate of 0.4 μL/min and a temperature of 42° C. on binary pump systems using 0.1% formic acid in water as mobile phase A and 0.1% formic acid in ACN as mobile phase B. Four μL of each sample were loaded onto the column at a flow rate of 0.5 μL/min for 10 min and separated using a binary gradient of 0.5-5% in 0.5 min, 5-20% B in 26.5 min, 20-25% B in 10 min, 25-38.5% B in 8 min, and 38.5-95% B in 1 min. The TSQ Vantage was operated with ion spray voltages of 2,400 V and a capillary inlet temperature of 370° C. Tube lens voltages were obtained from automatic tuning and calibration without further optimization. Both Q1 and Q3 were set at unit resolution of 0.7 FWHM and Q2 gas pressure was 1.5 mTorr. A scan width of 0.002 m/z was used. Because of the large number of transitions to be scanned, a scheduled SRM method with RT window set to be 4 min and cycle time of 1 second was used.

Configuration 2: 16 peptides (16 proteins) in the remaining 233 samples. For the remaining 233 samples in the cohort, the number of protein candidates was reduced from 52 to 16. In order to achieve similar or even higher sensitivity with higher throughput, only the target-containing fractions (roughly 16 fractions) were selected during PRISM via online SRM monitoring of the heavy peptides instead of concatenating into 24 fractions, for the second dimension LC-SRM analysis; a faster LC gradient was also used for the LC-SRM analysis of the PRISM fractions. Briefly, separations were performed at a flow rate of 0.4 μL/min and a temperature of 42° C. on binary pump systems using 0.1% formic acid in water as mobile phase A and 0.1% formic acid in ACN as mobile phase B. Four μL of each sample were loaded onto the column at a flow rate of 0.5 μL/min for 10 min and separated using a binary gradient of 0.5-10% B in 0.5 min, 10-15% B in 1 min, 15-25% B in 6 min, 25-35% B in 3 min, and 35-95% B in 2 min. A non-scheduled SRM method with dwell time of 10 ms for each transition was used for analysis of the much smaller set of 16 peptides, while other MS conditions remained the same.

Assay consistency evaluation between the two PRISM-SRM configurations. In order to evaluate the consistency between the first and second PRISM-SRM configurations, 3 individual samples from the first set of 105 were used and analyzed to quantify the final 16 peptides (of 16 proteins) using both configurations. The average measurement variations of 3 samples between two configurations for all the 16 peptides are between 2% and 29% with a median of 5% (data not shown), which demonstrated the consistency in peptide quantification between two PRISM-SRM configurations.

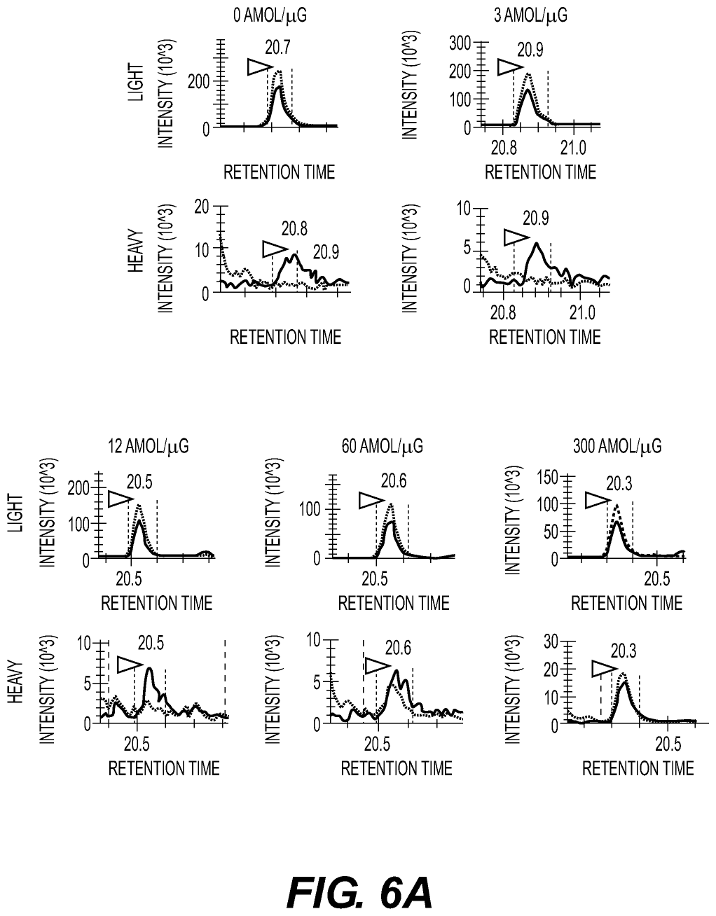

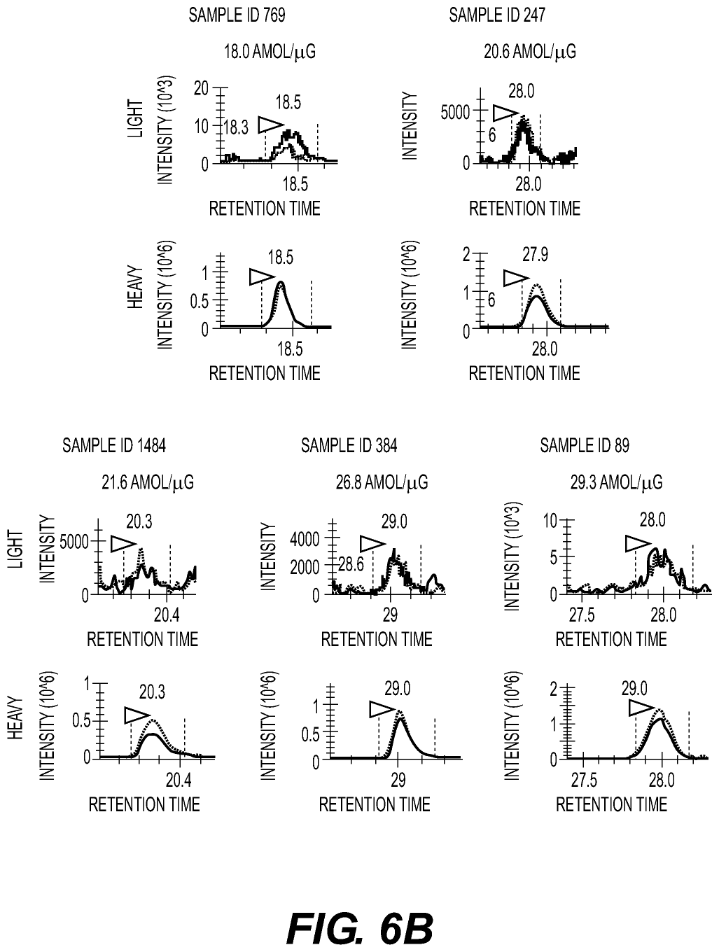

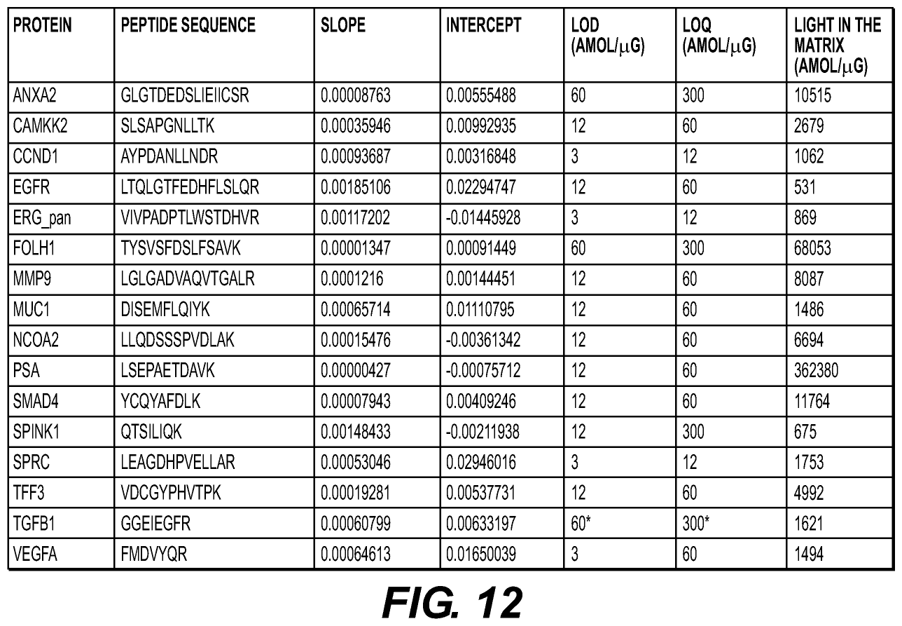

Response curves for the PRISM-SRM assays. The sensitivity and linearity of the PRISM-SRM assay were determined by measuring heavy isotope-labeled peptide standards spiked into a sample pooled from all the remaining study samples to final concentrations of 0, 0.6, 3, 12, 60, 300, 1,500, 3,000, 6,000, 12,000, 24,000, and 48,000 amol/μg. Each of the above samples were subjected to the same PRISM-SRM workflow as indicated for Configuration 2 with 3 injection replicates. The response curves of each peptide were generated using the heavy-over-light peak area ratios and the heavy peptides concentration as indicated above. The signal-to-noise ratio (S/N) was calculated by the peak apex intensity over the highest background noise in a retention time region of ±15 s for the target peptides. The background noise levels were conservatively estimated by visually inspecting chromatographic peak regions. The lower limit of detection (LOD) and quantification (LOQ) were defined as the lowest concentration point of target proteins at which the S/N of surrogate peptides was at least 3 and 10, respectively. Additionally, LOQs also require a coefficient of variation (CV) less than 20%. The final LOD and LOQ values of each assay are provided in FIG. 12. Given the significant interference for the heavy peptide transitions of TGFB1 peptide GGEIEGFR (SEQ ID NO: 8; shown in FIGS. 6A-6B), the LOD and LOQ of GGEIEGFR (SEQ ID NO: 8) cannot be accurately determined; however, to ensure that the S/N ratios of the endogenous (light) peptides are acceptable, manual inspection was used.

SRM data analysis. The raw data acquired on the TSQ Vantage triple quadrupole MS were imported into Skyline software [3] for visualization and quantification. The total peak area ratio between endogenous peptides and their heavy isotope labeled peptide standards was used for quantification. Peak detection and integration were based on two criteria: 1) the same retention time and 2) approximately the same relative peak intensity ratios across multiple transitions between endogenous peptide and heavy isotope labeled peptide standards. All data were manually inspected to ensure correct peak detection and accurate integration.

Endogenous concentration calculation. The final endogenous peptide concentration (amol/μg) for all the samples was calculated using the response curves. The steps used to calculate the final concertation of peptides in the study samples are provided below.

Step 1. Fit the calibration curve using linear regression (Y=Slope*X+Intercept), where X is the heavy peptide concentration in amol/μg, and Y is the heavy over light peak area ratio (H/L). The final Slope and Intercept values are provided in FIG. 12.

Step 2. Calculate the light peptide concentration of each peptide in the matrix (C_{light in response curve}) using the response curve obtained above and data at three heavy peptides spike-in levels (300, 1,500, 3,000 amol/μg), and obtain the average of calculated light peptide concentrations (amol/μg).

Step 3. Calculate the final endogenous peptide concentration in the study samples (C_{endogenous in sample}) using the Slope, Intercept, and C_{light in response curve} of the response curves. The equation is as follows:

$${c}_{endogenous in sample} = ( \frac{L / H {Ratio}_{in sample}}{{c}_{light in resposnse curve} / {c}_{heavy in sample}} - Intercept ) / slope$$

Where L/H Ration_{in sample} is the light over heavy peptide peak area ratio obtained directly from PRISM-SRM measurements, and C_{heavy in sample} is the heavy peptide concentration spiked in the study samples (amol/μg).

Statistical analysis. Analysis of variances (ANOVA) and Kruskal-Wallis test were used to examine the differences of distribution of continuous variables (age at diagnosis, follow up time, etc.) across event groups (non-event, BCR and metastasis), while Chi-square testing or Fisher exact test were used to evaluate the associations of categorical clinic-pathological variables (NCCN risk strata, pathological T stage, GG, surgical margin status) across event groups.

Unadjusted Kaplan-Meier survival analysis and log-rank testing were used to examine the DM-free survival curves stratified by the protein classifier cut point. Whisker boxplots were used to show differences in protein marker distribution across the 3 event groups (metastasis, BCR, non-events).

Univariable logistic regression and ROC curve analysis were used to identify which protein markers were significantly associated with high GG (GG3-4 versus GG1-2), metastasis (vs. non-event), or BCR (vs. non-event). The significance level was adjusted using the Bonferroni method: given 16 protein markers analyzed, the alpha level was adjusted to p=0.05/16=0.003125.

Due to significant correlations observed between several markers predicting metastasis, BCR, and/or high GG, a principle components analysis (PCA) was conducted. Those significant markers drawn from above univariable analyses to form principle components (PCs), PC was included in the further multivariable logistic regression analysis for predicting metastasis and BCR as a covariate, in combining with clinical or pathological variables. Only the first PC in each model had Eigenvalue greater than 1, which was kept in the multivariable analyses.

Using multivariable logistic regression analysis, ROC curves were generated to evaluate prediction accuracy based on AUC statistics of each set of protein marker “panels” for each outcome: metastasis, BCR, and high GG. For each ROC curve analysis, the protein panel was examined in combination with standard of care (SOC) variables versus the SOC variables alone. Two “base” models comprised of SOC variables were examined: 1) a biopsy “base” model, including patient age at PCa diagnosis, self-reported race, and NCCN risk stratum, and 2) a pathology “base” model, including age at PCa diagnosis, self-reported race, pathological T stage, GG, and surgical margin status. Because none of those patients with GG1-3 ever developed metastasis, they were removed from the metastasis pathology “base” model ROC curve analysis, leaving only two categories for GG (5 vs. 4). Comparisons between AUC values were conducted using maximum likelihood ratio testing.

Multivariable Cox proportional hazards (PH) models were constructed to examine distant metastasis-free survival and BCR-free survival as a function of both the protein marker panels specific to each study endpoint (metastasis, BCR), adjusting for the biopsy “base” model covariates, followed by adjustment for the pathologic “base” model covariates (a total of 4 Cox PH models).

All 95% confidence intervals (CI) for AUC values were constructed using non-parametric, bootstrapping method with 1,000 replicates.

Further, two composite variables were created from individual protein markers: 1) a SPARC score=SPARC protein level/PSA protein level and 2) a TGFB1 score=TGFB1 protein level/PSA protein level. Both the biopsy “base” and pathology “base” ROC models were repeated, comparing base models alone versus in combination with each composite variable (FIGS. 6A-6B).

All statistical analysis was performed using SAS version 9.4 (North Carolina), and statistical significance was p<0.05 (except for univariable analysis of individual protein markers, described previously).

### Example 2

### Study Design and Study Cohort

A retrospective cohort study was conducted using the Water Reed National Military Medical Center (WRNMMC) prostate cancer Biospecimen Repository linked to the Center for Prostate Disease Research (CPDR) Multi-center National Database. In brief, specimens in the WRNMMC Biospecimen Repository were collected from PCa patients who underwent RP at WRNMMC and who provided informed consent to donate prostatectomy specimens to the repository and enrollment in the CPDR Multi-center National Database clinical data repository. The Multi-center National Database contains detailed demographic, clinical, treatment, pathologic, and outcomes information. Further details about these databases have been reported previously (Sun and Vaughn, Semin Urol Oncol., 19 (3):186-93, 2001). Both repositories and multi-center national database have Institutional Review Board (IRB) approval at the WRNMMC and the Uniformed Services University of the Health Sciences (USUHS), respectively.

### Example 3

### Protein Marker Selection Process

The initial list of candidate markers included a 151-gene panel, which included 1) 27 PCa-specific gene fusions, 2) 44 genes over- or under-expressed in PCa versus matched benign epithelium or that were prostate tissue-specific, 3) 34 genes implicated in or associated with PCa progression, 4) 25 general cancer associated genes, and 5) five genes encoding the ETS-family of transcription factors. In addition, five prostate stroma or epithelium-specific genes were selected as control markers, and 11 housekeeping genes were included for biological normalization purposes. These 151 genes were incorporated into a NanoString Code Set, which was used to identify differential mRNA expression. Based on the transcriptome results, the original list of 151 decreased to 52 candidates for SRM assays and the targeted proteomics measurements, as described in Example 1.

### Example 4

### Phased Evaluation and Identification of Study Cohorts

Candidate biomarkers were evaluated in three steps that involved an incremental construction of the overall study cohort. First, a feasibility study was conducted in which 30 patients were selected to examine the proteomics assay sensitivity for the 52-markers in FFPE prostatectomy tissue specimens, including 10 patients who developed distant metastasis after RP, 10 patients who did not have metastasis but developed BCR, and 10 non-event patients who had at least 10 years follow up with no evidence of any disease progression after RP. Using the state-of-the-art, antibody-independent PRISM-SRM method allows for much higher sample loading (for example, 70× in the current study) as well as highly effective peptide enrichment and significantly reduced sample complexity that provided much higher sensitivity and is, thus, well-suited for the detection of genomic biomarker candidates at the protein level. As expected, compared to the regular LC-SRM, which detected 21 proteins, the PRISM-SRM method allowed for detection of a much larger list of 42 proteins. Ten of the original 52 markers showed poor sensitivity in the FFPE prostatectomy specimens. These proteins were excluded from further analysis, leaving 42 markers for subsequent testing.

In the second phase, a test cohort was created, which included 105 patients (48 non-events, 37 BCR, 20 DM), who were selected to test the association between the 42 protein markers and PCa progression. Non-events were based on a minimum of 10 years follow-up after RP with approximately a 3:2:1 ratio across event groups. In this cohort, 16 protein markers demonstrated a statistically significant difference in distribution across these three event groups, including: ANXA2, CAMKK2, CCND1, EGFR, ERG_pan, FOLH1, MMP9, MUC1, NCOA2, PSA, SMAD4, SPINK1, SPARC, TFF3, TGFB1, and VEGFA.

In the final study phase, the PRISM-SRM assays were reconfigured to measure only these 16 proteins, testing their ability to discriminate event status in an additional 233 patients that were selected to maintain comparable ratios across the 3 event groups, providing a summary n=338 (161:124:53 for non-events:BCR events:metastatic events).

Details for the PRISM-SRM assays are provided in FIGS. 11A-12. The linearity and interference issues have been carefully evaluated and demonstrated in FIGS. 5A-5P and FIGS. 6A-6B, respectively.

### Example 5

### Biomarker Performance in the Combined Cohort

In the final study cohort of 338 patients, 53 (15.7%) experienced distant metastasis, 124 (36.7%) progressed to BCR, and 161 (47.6%) had no evidence of disease progression after a minimum of 10 years of follow-up time (Table 1). Median patient age at PCa diagnosis and post-RP follow-up times were 59.5 and 12.5 years, respectively. Median time from RP to BCR and metastasis was 1.7 and 6.7 years, respectively for those who did progress.

TABLE 1
Distribution of clinico-pathological variables across event groups.

| Variable | All | | Non-event | | BCR | | Metastasis | | P value |
|---|---|---|---|---|---|---|---|---|---|
| N | 338 | | 161 | | 124 | | 53 | | |
| Age at diagnosis (yr) | | | | | | | | | |
| Mean (SD) | 59.5 | (7.7) | 59.0 | (8.1) | 59.2 | (7.7) | 61.7 | (5.9) | 0.0897 |
| Time from Dx to RP (mos) | | | | | | | | | |
| Median (range) | 2.3 | (0.2-21) | 2.2 | (0.2-21) | 2.5 | (0.2-9) | 2.0 | (0.7-10) | 0.4689 |
| Race | | | | | | | | | |
| AA | 120 | (35.6) | 55 | (34.2) | 48 | (39.0) | 17 | (32.1) | |
| CA & Other | 217 | (64.4) | 106 | (65.8) | 75 | (61.0) | 36 | (67.9) | 0.5882 |
| PSA at diagnosis (ng/mL) | | | | | | | | | |
| <10 | 262 | (78.0) | 133 | (83.6) | 90 | (72.6) | 39 | (73.6) | |
| 10-20 | 59 | (17.6) | 25 | (15.7) | 25 | (20.2) | 9 | (17.0) | |
| >20 | 15 | (4.5) | 1 | (0.6) | 9 | (7.3) | 5 | (9.4) | 0.0062 |
| Clinical T stage | | | | | | | | | |
| T1-T2a | 274 | (82.0) | 134 | (85.4) | 107 | (86.3) | 33 | (62.3) | |
| T2b-T2c | 52 | (15.6) | 22 | (14.0) | 15 | (12.1) | 15 | (28.3) | |
| T3a-T4 | 8 | (2.4) | 1 | (0.6) | 2 | (1.6) | 5 | (9.4) | 0.0005 |
| Biopsy grade | | | | | | | | | |
| 6 or less | 182 | (58.3) | 100 | (70.9) | 68 | (57.1) | 14 | (26.9) | |
| 7 | 95 | (30.4) | 35 | (24.8) | 41 | (34.4) | 19 | (36.5) | |
| 8-10 | 35 | (11.2) | 6 | (4.3) | 10 | (8.4) | 19 | (36.5) | <.0001 |
| NCCN risk | | | | | | | | | |
| Low | 125 | (40.6) | 69 | (50.7) | 46 | (38.3) | 10 | (19.2) | |
| Intermediate | 134 | (43.5) | 59 | (43.4) | 55 | (45.8) | 20 | (38.5) | |
| High | 49 | (15.9) | 8 | (5.9) | 19 | (15.8) | 22 | (42.3) | <.0001 |
| Pathological T stage | | | | | | | | | |
| pT2 | 174 | (52.6) | 119 | (74.4) | 46 | (37.4) | 9 | (18.8) | |
| pT3-4 | 157 | (47.4) | 41 | (25.6) | 77 | (62.6) | 39 | (81.2) | <.0001 |
| GG | | | | | | | | | |
| GG1 | 31 | (9.3) | 18 | (11.2) | 13 | (10.6) | 0 | | |
| GG2 | 105 | (31.6) | 77 | (48.1) | 27 | (22.0) | 1 | (2.0) | |
| GG3 | 6 | (1.8) | 2 | (1.2) | 4 | (3.2) | 0 | | |
| GG4 | 124 | (37.4) | 54 | (33.8) | 49 | (39.8) | 21 | (42.9) | |
| GG5 | 66 | (19.9) | 9 | (5.6) | 30 | (24.4) | 27 | (55.1) | <.0001 |
| Surgical margin | | | | | | | | | |
| Negative | 209 | (63.7) | 126 | (79.2) | 62 | (51.2) | 21 | (43.8) | |
| Positive | 119 | (36.3) | 33 | (20.8) | 59 | (48.8) | 27 | (56.2) | <.0001 |
| FU (mos) | | | | | | | | | |
| Median (range) | 150 | (18-253) | 156 | (121-252) | 129 | (18-229) | 124 | (24-253) | <.0001 |

Several notable differences were observed across the three event groups, including poorer clinical features at time of PCa detection and poorer pathological features at time of RP for those who ultimately experienced disease progression (i.e., BCR and metastasis). This included higher pathologic grade at RP; among those who developed metastasis, 98% had Grade Group (GG) 4-5 at RP compared to 64% of those who developed BCR and only 39% of those in the non-event group (p<0.0001). No racial differences were noted across event status (p=0.59).

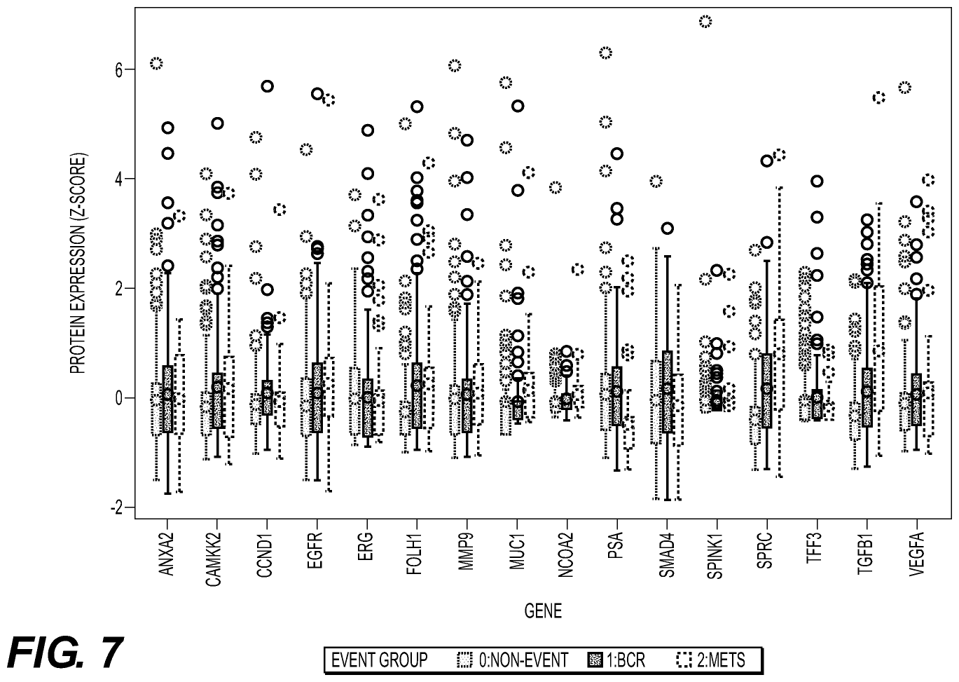

Area under the curve (AUC) statistics are shown in Table 2 for each of the selected 16 protein markers for predicting metastasis (yes versus no) and BCR (yes versus no) events as well as discriminating high GG (4-5 versus 1-3). Bonferroni correction for multiple comparisons (p=0.05/16=0.0031) was used to ascertain statistical significance. The protein expression levels across the three event groups are visualized using whistker boxplot in FIG. 7. Three proteins that were statistically significant predictors across all 3 endpoints (i.e., metastasis, BCR, GG) included FOLH1, SPARC, and TGFB1. In addition, PSA was predictive of distant metastasis while CAMKK2, EGFR, and NCOA2 were also predictive of high GG.

TABLE 2
Individual AUC and P values of 16 proteins to
predict metastasis, BCR, or GG.

| | DM | | BCR vs. | | | |
|---|---|---|---|---|---|---|
| | vs. non-event | | non-event | | GG (3-5 vs 1-2) | |
| Gene | AUC | P value | AUC | P value | AUC | P value |
| ANXA2 | 0.535 | 0.741 | 0.538 | 0.341 | 0.499 | 0.692 |
| CAMKK2 | 0.591 | 0.051 | 0.604 | 0.009 | 0.667 | <.001 |
| COND1 | 0.532 | 0.166 | 0.624 | 0.037 | 0.592 | 0.034 |
| EGFR | 0.628 | 0.012 | 0.578 | 0.035 | 0.653 | <.001 |
| ERG | 0.543 | 0.668 | 0.546 | 0.830 | 0.482 | 0.708 |
| FOLH1 | 0.653 | 0.001 | 0.627 | <.001 | 0.657 | <.001 |
| MMP9 | 0.562 | 0.518 | 0.511 | 0.770 | 0.554 | 0.643 |
| MUC1 | 0.570 | 0.461 | 0.474 | 0.603 | 0.506 | 0.200 |
| NCOA2 | 0.637 | 0.095 | 0.613 | 0.225 | 0.670 | 0.001 |
| PSA | 0.730 | 0.001 | 0.529 | 0.955 | 0.608 | 0.005 |
| SMAD4 | 0.511 | 0.622 | 0.526 | 0.092 | 0.521 | 0.383 |
| SPINK1 | 0.486 | 0.207 | 0.548 | 0.535 | 0.547 | 0.470 |
| SPRC | 0.800 | <.001 | 0.695 | <.001 | 0.715 | <.001 |
| TFF3 | 0.541 | 0.174 | 0.472 | 0.578 | 0.492 | 0.751 |
| TGFB1 | 0.788 | <.001 | 0.649 | <.001 | 0.705 | <.000 |
| VEGFA | 0.528 | 0.168 | 0.601 | 0.040 | 0.573 | 0.009 |

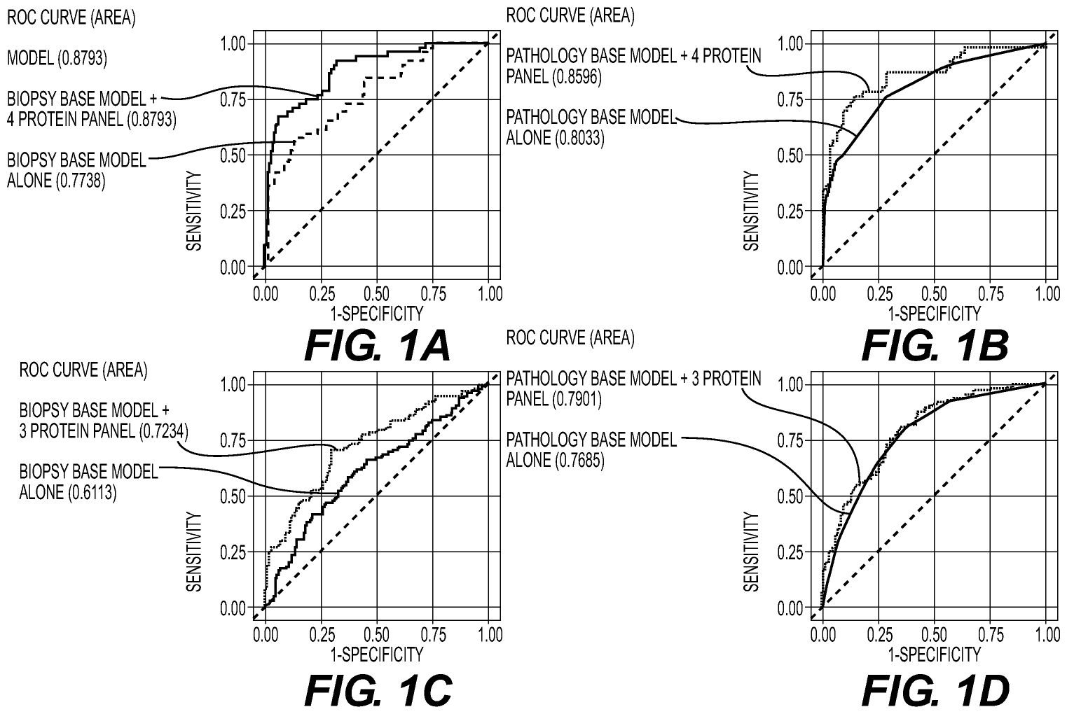

FIGS. 1A and 1B display receiver operating characteristic (ROC) curve analyses for distant metastasis with consideration of the biopsy standard of care (SOC) model (including age, race and NCCN-risk strata) (FIG. 1A) and pathology SOC model (including pathological T stage, GG and surgical margin status) (FIG. 1B) for the protein panel of 4 markers that individually demonstrated statistical significance for predicting distant metastasis (FOLH1, SPARC, TGFB1, and PSA). When modeling the protein panel in combination with the biopsy base model, the 4-protein panel significantly improved the AUC curve (p=0.002); however, when combined with the pathology base model, the improvement was not statistically significant (p=0.055).

Similarly, in FIGS. 1C and 1D, ROC curve analyses were examined for BCR with consideration of the biopsy base model (FIG. 1C) and the pathology base model (FIG. 1D) for the protein panel of 3 markers that individually demonstrated statistical significance for predicting BCR (FOLH1, SPARC, and TGFB1). When modeling the protein panel in combination with the biopsy base model, the 3-protein panel significantly improved the AUC curve (p=0.003), but when combined with the pathology base model, the improvement was not statistically significant (p=0.050).

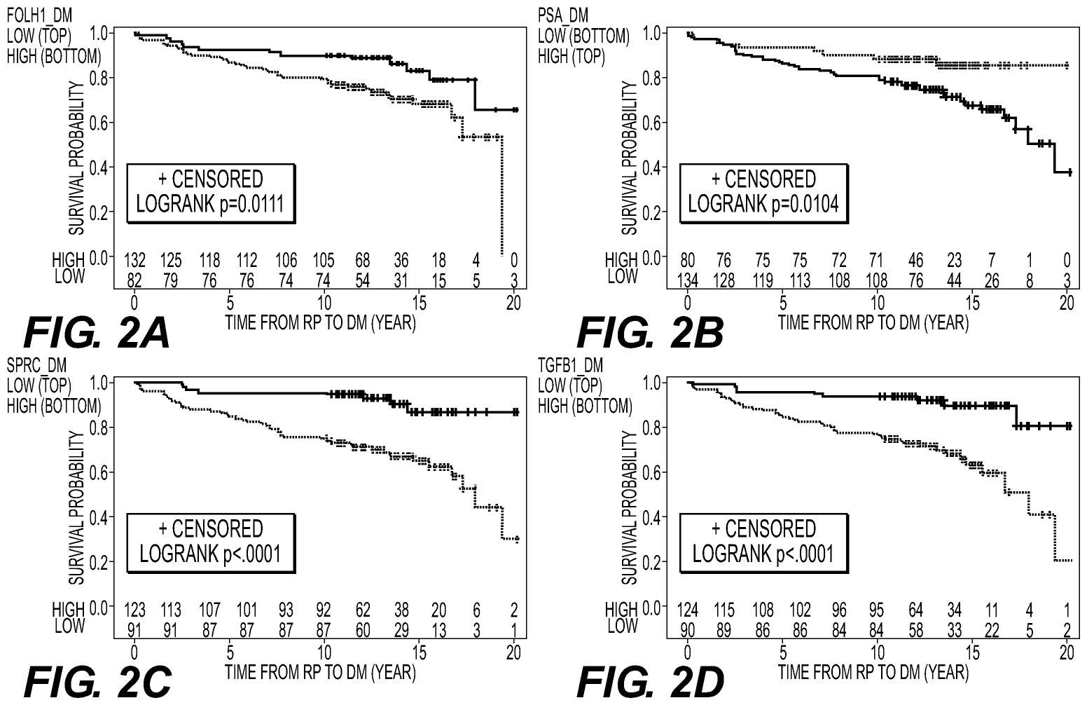

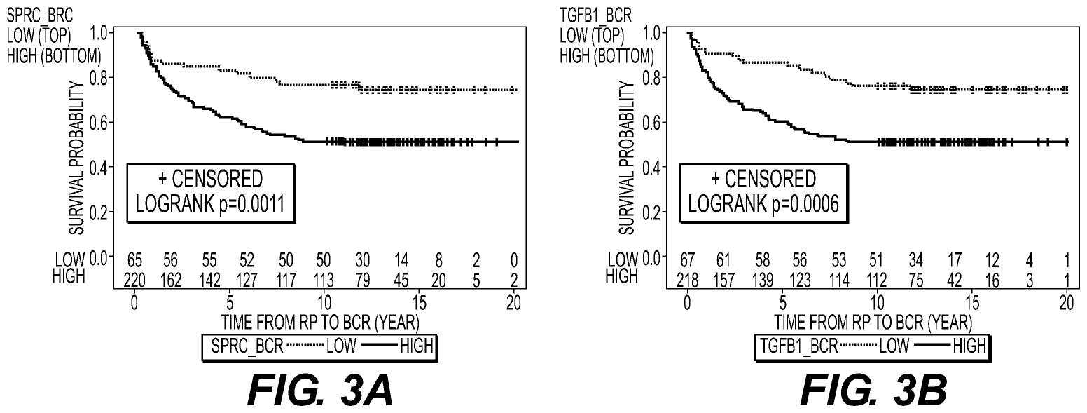

Optimal cut-off points for each protein were then identified (Tables 3A-3B). Only markers with the highest sensitivity among the cut points which could achieve at least 70% negative predictive value (NPV) and 30% specificity were utilized in subsequent stratified Kaplan Meier (KM) estimation curve analysis (FIGS. 2A-2D and 3A-3B). In unadjusted KM analyses, all 4 protein markers were individually predictive of distant metastasis free survival (FIGS. 2A-2D). Similarly, for both protein markers of BCR that met the criteria set forth in Table 2, each was a significant predictor of BCR-free survival (FIGS. 3A-3B).

TABLE 3A
Cut off of each protein and boostrapped 95% CI
for predicting distant metastasis.

| Protein | Cut point | 95% CI | Sensitivity | Specificity | PPV | NPV |
|---|---|---|---|---|---|---|
| FOLH1 | −0.54 | −0.55, −0.53 | 0.731 | 0.419 | 0.325 | 0.803 |
| PSA | −0.12 | −0.15, −0.08 | 0.827 | 0.412 | 0.350 | 0.862 |
| SPARC | −0.53 | −0.55, −0.52 | 0.865 | 0.522 | 0.409 | 0.910 |
| TGFB1 | −0.50 | −0.52, −0.48 | 0.846 | 0.493 | 0.389 | 0.893 |

*Optimal cut off was chosen a point value with the highest sensitivity among the cut points which satisfy at least 80% NPV and 40% specifisity.

TABLE 3B
Cut off of each protein and boostrapped 95% CI for predicting BCR.

| Protein | Cut point | 95% CI | Sensitivity | Specificity | PPV | NPV |
|---|---|---|---|---|---|---|
| SPARC | −0.74 | −0.75, −0.72 | 0.874 | 0.301 | 0.523 | 0.732 |
| TGFB1 | −0.71 | −0.73, −0.69 | 0.866 | 0.309 | 0.523 | 0.724 |

*Optimal cut off was chosen a point value with the highest sensitivity among the cut points which satisfy at least 70% NPV and 30% specifisity.

Finally, multivariable Cox proportional hazards analyses were performed for DM-free survival (Table 4A) and BCR-free survival (Table 4B) with markers that achieved the criteria established in Tables 3A-3B, adjusting first for the base model variables, then separately for the pathology base model variables. In the biopsy base models, both higher NCCN risk strata and higher SPARC cut-off points consistently predicted poor outcome (DM- and BCR-free survival). However, TGFB1 was not a significant predictor of either study outcome in either the biopsy or pathology base models.

TABLE 4A
Multivariable Cox model predict distant metastasis for adding protein
panel (FOLH1, PSA, SPARC and TGFB1) to biopsy or pathology base model.

| | Panel + Biopsy base model: | Panel + Pathology base model: |
|---|---|---|
| Variable | OR (95% CI); P | OR (95% CI); P |
| Age at diagnosis | 1.04 (0.99-1.08); 0.055 | 1.04 (0.98-1.09); 0.172 |
| Race: AA vs CA | 0.70 (0.38-1.30); 0.262 | 0.66 (0.33-1.32); 0.240 |
| NCCN risk | | |
| Intermediate vs Low | 1.68 (0.75-3.76); 0.206 | — |
| High vs low | 5.57 (2.48-12.53); <.001 | — |
| Pathological T stage: T3 vs T2 | — | 3.36 (1.54-7.34); 0.002 |
| GG: 5 vs 1-4 | — | 4.39 (2.29-8.44); <.001 |
| Surgical margin: pos vs neg | — | 1.29 (0.69-2.41); 0.423 |
| FOLH: ≥−0.54 vs. <−0.54 | 1.25 (0.62-2.52); 0.529 | 1.34 (0.62-2.88); 0460 |
| PSA: ≥−0.12 vs. <−0.12 | 0.58 (0.29-1.19); 0.138 | 0.62 (0.30-1.28); 0.198 |
| SPARC: ≥−0.53 vs. <−0.53 | 2.51 (1.08-5.82); 0.031 | 3.14 (1.09-9.08); 0.034 |
| TGFB1: ≥−0.50 vs. <−0.50 | 2.21 (0.99-4.96); 0.053 | 1.03 (0.42-2.52); 0.983 |

TABLE 4B
Multivariable Cox model predict BCR for adding protein panel (FOLH1,
SPARC and TGFB1) to biopsy or pathology base model.

| | Panel + Biopsy base model: | Panel + Pathology base model: |
|---|---|---|
| Variable | OR (95% CI); P | OR (95% CI); P |
| Age at diagnosis | 1.00 (0.98-1.02); 0.973 | 0.99 (0.97-1.02); 0.626 |
| Race: AA vs CA | 1.10 (0.76-1.60); 0.620 | 1.19 (0.81-1.74); 0.369 |
| NCCN risk | | |
| Intermediate vs Low | 1.08 (0.72-1.62); 0.710 | — |
| High vs low | 10.99 (1.15-3.46); 0.013 | — |
| Pathological T stage: T3 vs T2 | — | 2.10 (1.36-3.25); 0.001 |
| GG: 3-5 vs 1-2 | — | 1.58 (1.04-2.39); 0.030 |
| Surgical margin: pos vs neg | — | 1.75 (1.19-2.58); 0.004 |
| SPARC: ≥−0.74 vs. <−0.74 | 2.00 (1.27-3.16); 0.002 | 1.50 (0.92-2.44); 0.100 |
| TGFB1: ≥−0.71 vs. <−0.71 | 1.32 (0.87-2.00); 0.195 | 1.02 (0.65-1.58); 0.940 |

### Example 6

### Development of Protein Classifier: Training and Testing Set Validation Analyses

Results from 214 patients (53 distant metastasis and 161 non-events) were used to develop a proteomic classifier to predict distant metastasis. This 214-patient cohort was randomly split into training and testing data sets (70% vs 30%) (Journal of Hydrology. v529:1060-1069, 2015). The comparison of distribution of clinical-pathological variables between training and testing cohorts are provided in Table 5. There was no significant difference in the distribution of clinical-pathological variables between training and testing cohorts, except that the testing cohort had slightly shorter median follow up time than training cohort (p=0.049).

TABLE 5
Clinical variable distribution between training and testing cohorts.

| | | | | | | Pathological | | | | |
|---|---|---|---|---|---|---|---|---|---|---|
| Clinical Variable | Training | | Testing | | P value | variable | Training | | Testing | P value |
| N | 149 | | 65 | | | 149 | 65 | | | |
| Age at diagnosis | | | | | | Pathological | | | | |
| (yr) | | | | | | T stage | | | | |
| Mean (SD) | 59.4 | (7.8) | 60 | (7.5) | | pT2 | 91 | (61.9) | 37 (60.7) | |
| Median (range) | 60 | (35-75) | 62 | (42-75) | 0.419 | PT3 | 56 | (38.1) | 24 (39.3) | 0.866 |
| Race | | | | | | GG | | | | |
| AA | 53 | (35.6) | 19 | (29.2) | | GG1-2 | 125 | (84.5) | 48 (78.7) | |
| CA&other | 96 | (64.4) | 46 | (70.8) | 0.367 | GG3-5 | 23 | (15.5) | 13 (21.3) | 0.316 |
| PSA at | | | | | | Surgical | | | | |
| diagnosis | | | | | | margin | | | | |
| (ng/mL) | | | | | | status | | | | |
| <10 | 116 | (78.9) | 56 | (86.2) | | Negative | 102 | (69.9) | 45 (73.8) | |
| 10-19.99 | 26 | (17.7) | 8 | (12.3) | | Positive | 44 | (30.1) | 16 (26.2) | 0.573 |
| 20 or above | 5 | (3.4) | 1 | (1.5) | 0.2 | Upgrade | | | | |
| Clinical T stage | | | | | | No | 43 | (55.1) | 19 (52.8) | |
| T2-T2a | 112 | (76.7) | 55 | (85.9) | | Yes | 35 | (44.9) | 17 (47.2) | 0.815 |
| T2b-c | 29 | (19.9) | 8 | (12.5) | | Time from | | | | |
| T3a or above | 5 | (3.4) | 1 | (1.6) | 0.368 | Dx to RP | | | | |
| Biopsy Gleason | | | | | | (mos) | | | | |
| sum | | | | | | Mean (SD) | 2.7 | (2.1) | 3.1 (2.7) | |
| 6 or less | 78 | (59.1) | 36 | (59.0) | | Median | 2.1 | (0.2-21) | 2.2 (0.4-15) | 0.514 |
| 7 | 39 | (29.5) | 15 | (24.6) | | (range) | | | | |
| 8-10 | 15 | (11.4) | 10 | (16.4) | 0.644 | Follow up | | | | |
| NCCN risk | | | | | | after RP (mos) | | | | |
| Low | 20 | (15.6) | 10 | (16.7) | | Mean (SD) | 158.8 | (40.0) | 148.9 (46.5) | |
| Intermediate | 56 | (43.8) | 23 | (38.3) | | Median | 157 | (30-252) | 145 (24-253) | 0.049 |
| High | 52 | (40.6) | 27 | (45.0) | 0.766 | (range) | | | | |
| | | | | | | Event group | | | | |
| | | | | | | Non-event | 116 | (77.8) | 45 (69.2) | |
| | | | | | | DM | 33 | (22.2) | 20 (30.8) | 0.181 |

The training cohort consisted of 149 patients (33 DM, 116 non-events), and the testing cohort consisted of 65 patients (20 DM, 45 non-events). The comparison of distribution of clinical-pathological variables across event groups among these two cohorts are provided in Table 6. The NCCN risk strata, pathological T stage, RP GG, and surgical margins status showed significant associations with distant metastasis in both the training and testing cohorts.

TABLE 6
Distributions of clinico-pathological variables between non-
event and DM groups among training and testing cohorts.

| | Training | | | Testing | | |
|---|---|---|---|---|---|---|
| Variable | Non-event | DM | P value | Non-event | DM | P value |
| Age at diagnosis (yr) | 116 | 33 | | 45 | 20 | |
| Mean (SD) | 58.6 (8.1) | 62.3 (5.6) | | 59.9 (8.0) | 60.5 (6.3) | |
| Median (range) | 59. (35-75) | 62 (50-74) | 0.015 | 62 (42-75) | 61 (46-70) | 0.937 |
| Race | | | | | | |
| AA | 43 (37.1) | 10 (30.3) | | 12 (26.7) | 7 (35.0) | |
| CA&other | 73 (62.9) | 23 (69.7) | 0.475 | 33 (73.3) | 13 (65.0) | 0.498 |
| PSA at diagnosis (ng/mL) | | | | | | |
| <10 | 93 (81.6) | 23 (69.7) | | 40 (88.9) | 16 (80.0) | |
| 10-19.99 | 20 (17.5) | 6 (18.2) | | 5 (11.1) | 3 (15.0) | |
| 20 or above | 1 (0.9) | 4 (12.1) | 0.015 | 0 | 1 (5.0) | 0.277 |
| Clinical T stage | | | | | | |
| 12-T2a | 94 (83.2) | 18 (54.6) | | 40 (90.9) | 15 (75.0) | |
| T2b-c | 18 (15.9) | 11 (33.3) | | 4 (9.1) | 4 (20.0) | |
| T3a or above | 1 (0.9) | 4 (12.1) | <.001 | 0 | 1 (5.0) | 0.138 |
| Biopsy Gleason sum | | | | | | |
| 6 or less | 69 (69.7) | 9 (27.3) | | 31 (73.8) | 5 (26.3) | |
| =7 | 28 (28.3) | 11 (33.3) | | 7 (16.7) | 8 (42.1) | |
| 8-10 | 2 (2.0) | 13 (39.4) | <.001 | 4 (9.5) | 6 (31.6) | 0.001 |
| NCCN risk | | | | | | |
| Low | 46 (48.4) | 6 (18.2) | | 23 (56.1) | 4 (21.0) | |
| Intermediate | 45 (47.4) | 11 (33.3) | | 14 (34.2) | 9 (47.4) | |
| High | 4 (4.2) | 16 (48.5) | <.001 | 4 (9.8) | 6 (31.6) | 0.005 |
| Pathological T stage | | | | | | |
| pT2 | 87 (75.0) | 4 (12.9) | | 32 (72.7) | 5 (29.4) | |
| PT3 | 29 (25.0) | 27 (87.0) | <.001 | 12 (27.3) | 12 (70.6) | 0.002 |
| GG* | | | | | | |
| GG1-4 | 110 (94.8) | 15 (46.9) | | 41 (93.2) | 7 (41.2) | |
| GG5 | 6 (5.2) | 17 (53.1) | <.001 | 3 (6.8) | 10 (58.8) | <.001 |
| Surgical margin status | | | | | | |
| Negative | 91 (79.1) | 11 (35.5) | | 35 (79.6) | 10 (58.8) | |
| Positive | 24 (20.9) | 20 (64.5) | <.001 | 9 (20.4) | 7 (41.2) | 0.115 |

*Since there were only 1 patient developed DM among GG1-3 patients, so for DM study, GG1-4 patients were combined to one group.

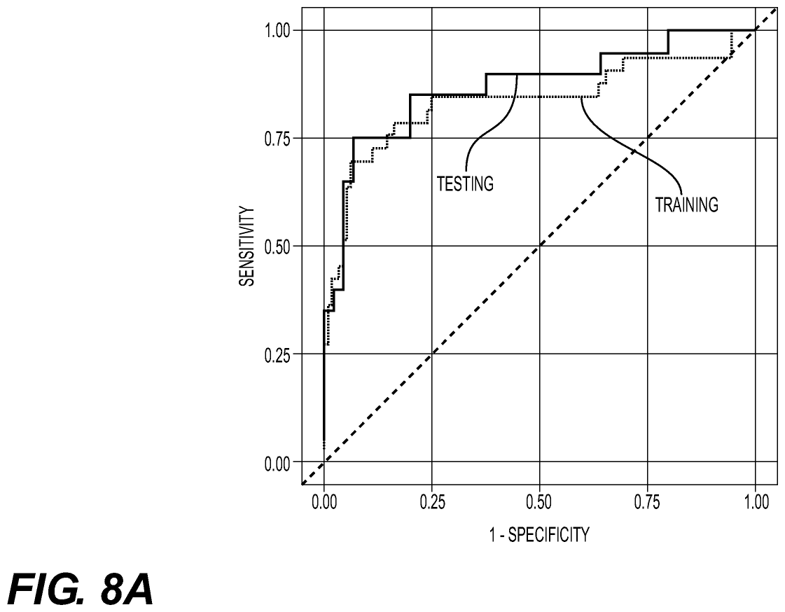

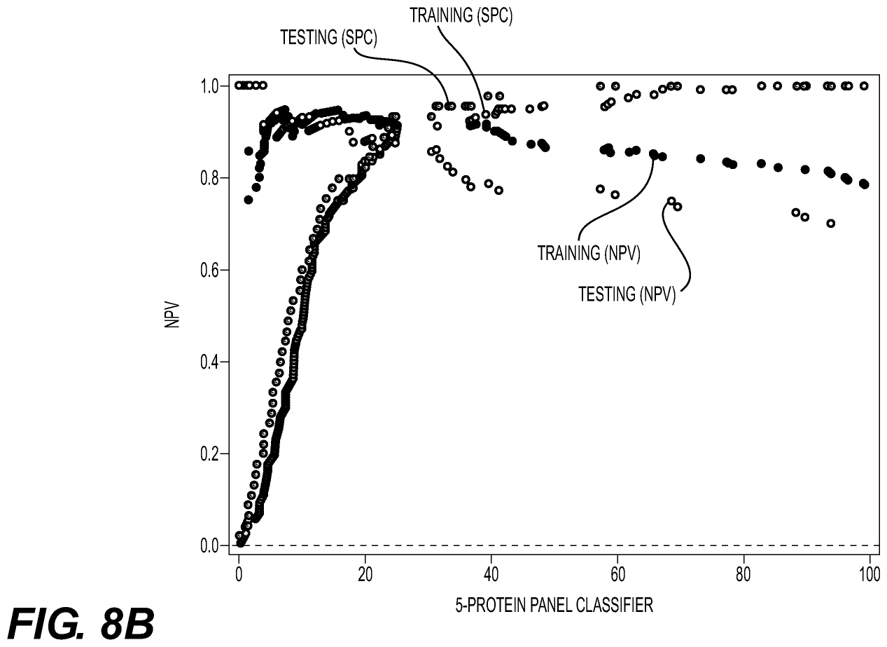

In the training cohort, univariable logistic regression analysis was used to select those biomarkers which are significantly predicting DM status (p<0.05 and AUC>0.65), including CAMKK2, FOLH1, PSA, SPARC and TGFB1. Next, multivariable logistic regression modeling was performed using those 5 proteins (CAMKK2, FOLH1, PSA, SPARC and TGFB1) to obtain parameter estimates for 5 proteins and to construct a 5-protein classifier in predicting DM, scaled from 0 to 100. Bootstrapped multivariable logistic regression (1000 replicates) was used to search for an optimal threshold for the protein classifier in predicting DM. The optimal threshold was defined as a cut-off point that maximizes sensitivity with at least a 90% NPV and at least a 35% specificity (SPC) (cite STM lung cancer paper). Finally, the protein classifier and its threshold were analyzed in the testing cohort. The protein classifier performance, in both the training and testing cohorts, is presented in FIGS. 8A-8B and Table 7. The AUCs of the 5-protein classifier for DM in both the training and testing cohorts were 0.84 and 0.87, respectively. At the cut-off point of 8.3, the protein classifier for DM generated a 92% NPV and a 90% sensitivity with a 53% specificity in testing cohort.

TABLE 7
Performance of optimal cutoff of DM risk score.

| 5-protein | Training | | | | | | | |
|---|---|---|---|---|---|---|---|---|
| Classifier | NPV | Sens | SPC | PPV | Testing | | | |
| Threshold | (95% CI) | (95% CI) | (95% CI) | (95% CI) | NPV | Sens | SPC | PPV |
| 8.3 | 0.913 | 0.879 | 0.362 | 0.282 | 0.923 | 0.900 | 0.533 | 0.462 |
| | (0.911- | (0.875- | (0.350- | (0.274- | | | | |
| | 0.915) | 0.883) | 0.374) | 0.289) | | | | |

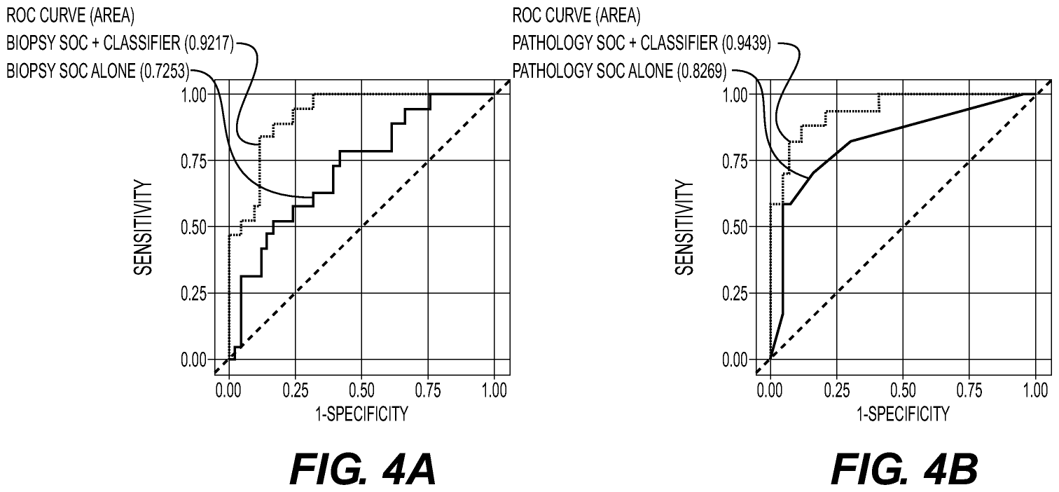

Multivariable logistic regression analysis, ROC analysis, and Mantel-Haenszel Chi-square tests were used to evaluate the prediction value of 5-protein classifier on DM by adding it to the biopsy and pathology SOC base models. Adding the protein classifier to the biopsy SOC model significantly enhanced the prediction value for DM with an increase in the AUC from 0.72 to 0.92 (p=0.001); similarly, adding the protein classifier to the pathology SOC model significantly increased DM prediction accuracy from AUC 0.83 to 0.94 (p=0.011) (FIG. 4).

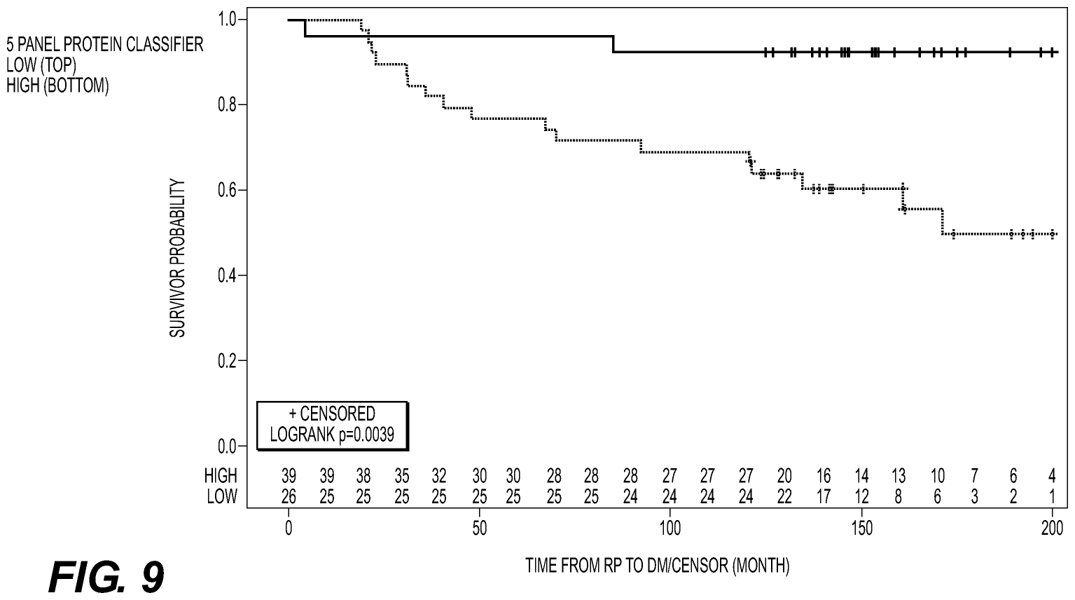

Unadjusted Kaplan-Meier survival analysis and log-rank testing were used to examine the DM-free survival curves stratified by the protein classifier cut-off point (FIG. 9). Patients with a high protein classifier value (≥8.3) had significantly worse DM-free survival than patients with a low protein classifier value (<8.3) (p=0.003).

The protein classifier (both in continuous and dichotomized at cutoff) was tested by adding it to both the biopsy SOC model and pathology SOC models using multivariable Cox proportional hazard analysis; the proportional hazards assumption of each covariate was checked and met. The results are presented in Table 8. After adjustment for biopsy SOC variables, patients with a high versus low protein classifier (≥8.3 vs. <8.3) had significantly greater risk of DM (HR=5.09, 95% CI: 1.11-23.38, p=0.036). When modeled as a continuous variable in multivariable analysis, the protein classifier showed significant independent prediction value for DM with adjustment for either biopsy SOC variables (HR=1.03, 95% CI: 1.02-1.05), p<0.001) (Table 8A) or pathology SOC variables (HR=1.02, 95% CI: 1.01-1.05, p=0.018) (Table 8B).

TABLE 8A
Multivariable Cox proportional hazard model predicting DM by adding
5-protein panel classifier to biopsy SOC in testing cohort.

| | | | | Model 2** | | |
|---|---|---|---|---|---|---|
| | Model 1* | | | | | P |
| Variable | HR | 95% CI | P value | HR | 95% CI | value |
| Age at diagnosis | 1.00 | 0.93-1.07 | 0.898 | 1.03 | 0.96-1.11 | 0.407 |
| Race (AA vs CA) | 0.94 | 0.33-2.74 | 0.916 | 1.59 | 0.54-4.64 | 0.396 |
| Risk (intermediate vs low) | 2.31 | 0.69-7.76 | 0.176 | 1.49 | 0.41-5.47 | 0.545 |
| Risk (high vs low) | 4.68 | 1.14-19.22 | 0.032 | 2.29 | 0.52-10.16 | 0.274 |
| 5-protein panel classifier* | 5.09 | 1.11-23.38 | 0.036 | 1.03 | 1.02-1.05 | <.001 |

*Model 1: Classifier was dichotomized at threshold of 8.3; **Model 2: Classifier was continuous

TABLE 8B
Multivariable Cox proportional hazard model predicting DM by adding
5-protein panel classifier to pathology SOC in testing cohort.

| | | | | Model 2** | | |
|---|---|---|---|---|---|---|
| | Model 1* | | | | | P |
| Variable | HR | 95% CI | P value | HR | 95% CI | value |
| Pathology T | 2.54 | 0.78-8.27 | 0.122 | 1.94 | 0.52-7.15 | 0.321 |
| (pT3 vs pT2) | | | | | | |
| GG (GG5 vs GG1-4) | 3.42 | 1.17-10.03 | 0.025 | 2.04 | 0.52-8.04 | 0.309 |
| Surgical margin | 1.31 | 0.47-3.68 | 0.603 | 1.23 | 0.42-3.57 | 0.705 |
| (Pos vs neg) | | | | | | |
| Risk score | 3.71 | 0.82-16.88 | 0.089 | 1.02 | 1.01-1.05 | 0.018 |
| (high vs low) | | | | | | |

*Model 1: Classifier was dichotomized at threshold of 8.3; **Model 2: Classifier was continuous

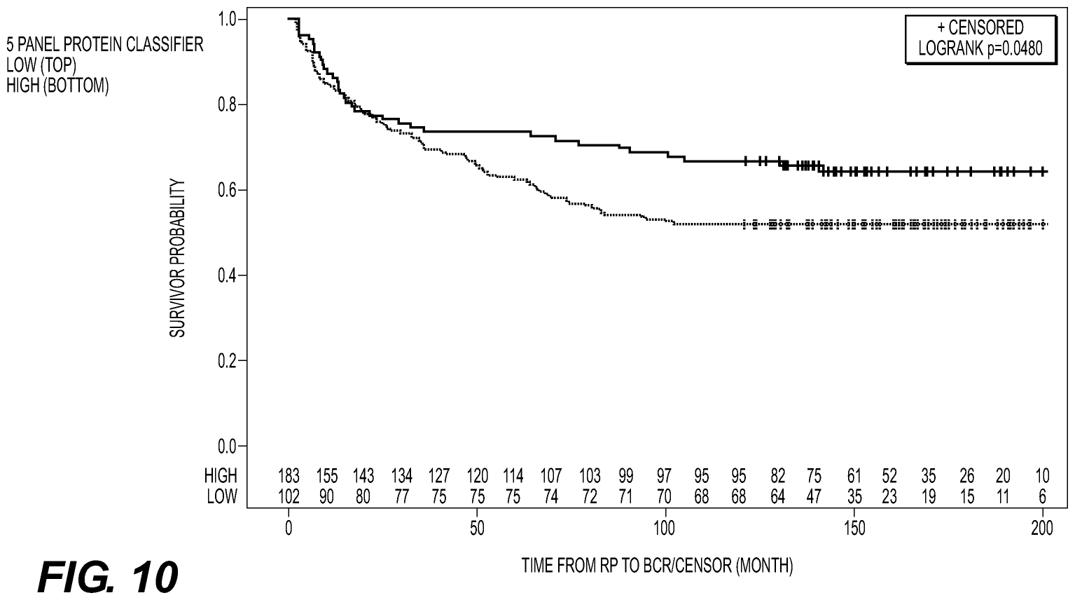

Similar to the description for DM, the BCR study cohort (n=285, 124 BCR and 161 non-events), was examined to validate the 5-protein panel classifier and its threshold in predicting BCR-free survival using unadjusted Kaplan-Meier survival analysis as well as multivariable Cox proportional hazard analysis using biopsy and pathology SOC models (see FIG. 10 and Table 9). Patients with a high protein classifier (≥8.3) had significantly worse BCR-free survival (p=0.048) than those with a low protein classifier (<8.3). The protein classifier (as a continuous variable) showed an independent prediction value on BCR when included in the biopsy SOC model (HR=1.02, 95% CI: 1.01-1.03, p<0.001). However, the classifier did not show significant prediction value in the pathology SOC model.

TABLE 9A
Multivariable Cox proportional hazard model predicting BCR
by adding 5-protein panel classifier to biopsy SOC in testing cohort.

| | | | | Model 2** | | |
|---|---|---|---|---|---|---|
| | Model 1* | | | | | P |
| Variable | HR | 95% CI | P value | HR | 95% CI | value |
| Age at diagnosis | 1.00 | 0.98-1.02 | 0.998 | 1.00 | 0.97-1.02 | 0.723 |
| Race (AA vs CA) | 1.18 | 0.81-1.71 | 0.399 | 1.20 | 0.82-1.74 | 0.346 |
| Risk (intermediate | 1.25 | 0.84-1.86 | 0.271 | 1.09 | 0.73-1.64 | 0.667 |
| vs low) | | | | | | |
| Risk (high vs low) | 2.35 | 1.36-4.07 | 0.002 | 1.78 | 1.01-3.15 | 0.045 |
| 5-protein panel | 1.25 | 0.83-1.86 | 0.284 | 1.02 | 1.01-1.03 | <.001 |
| classifier* | | | | | | |

*Model 1: Classifier was dichotomized at threshold of 8.3; **Model 2: Classifier was continuous

TABLE 9B
Multivariable
Cox proportional hazard model predicting DM by adding
3-protein DM risk score to pathology SOC in validation cohort.

| | | | | Model 2** | | |
|---|---|---|---|---|---|---|
| | Model 1* | | | | | P |
| Variable | HR | 95% CI | P value | HR | 95% CI | value |
| Pathology T | 2.48 | 1.64-3.75 | <.001 | 2.25 | 1.49-3.42 | <.001 |
| (pT3 vs pT2) | | | | | | |
| GG (GG5 vs | 2.21 | 1.43-3.42 | <.001 | 1.93 | 1.23-3.04 | 0.004 |
| GG1-4) | | | | | | |
| Surgical margin | 1.77 | 1.22-2.59 | <.001 | 1.67 | 1.14-2.45 | 0.008 |
| (Pos vs neg) | | | | | | |
| Risk score | 0.96 | 0.62-1.46 | 0.833 | 1.01 | 1.00-1.02 | 0.072 |
| (high vs low) | | | | | | |

*Model 1: Classifier was dichotomized at threshold of 8.3; **Model 2: Classifier was continuous

### Example 7

### Exhibit A

In view of the many possible embodiments to which the principles of the disclosure may be applied, it should be recognized that the illustrated embodiments are only examples of the disclosure and should not be taken as limiting in scope. Rather, the scope is defined by the following claims. We, therefore, claim all that comes within the scope and spirit of these claims.

1. (canceled)

2. A method of treating a subject with aggressive prostate cancer or at risk of developing aggressive prostate cancer, comprising:
administering a therapeutically effective amount of aggressive prostate cancer therapy to the subject having aggressive prostate cancer or at risk of developing aggressive prostate cancer, thereby treating the subject,
wherein prior to the administration of the therapeutically effective amount of aggressive prostate cancer therapy, the expression level of the aggressive prostate cancer-related molecules in a sample from the subject was determined to be increased as compared to a control, wherein the control represents expression of the aggressive prostate cancer-related molecules expected in a sample from a subject who does not have aggressive prostate cancer; and
wherein the aggressive prostate cancer-related molecules comprise folate hydro lase 1 (FOLH1), secreted protein acidic and rich in cysteine (SPARC), and transforming growth factor beta 1 (TGFB1).

3. (canceled)

4. The method of claim 2, wherein the aggressive prostate cancer-related molecules further comprise
calcium/calmodulin dependent protein kinase kinase 2 (CAMKK2), epidermal growth factor receptor (EGFR), nuclear receptor coactivator 2 (NCOA2), or prostate-specific antigen (PSA);
PSA;
CAMKK2, EGFR, and NCOA2; or
CAMKK2 and PSA.

5. The method of claim 2, wherein the subject with aggressive prostate cancer or at risk of developing aggressive prostate cancer comprises a subject at risk for post-surgical biochemical recurrent prostate cancer.

6. The method of claim 2, wherein the aggressive prostate cancer-related molecules further comprise PSA, and wherein the subject with aggressive prostate cancer or at risk of developing aggressive prostate cancer comprises a subject at risk for post-surgical distant metastatic prostate cancer.

7. The method of claim 2, wherein the aggressive prostate cancer-related molecules further comprise CAMKK2, EGFR, and NCOA2, and wherein the subject with aggressive prostate cancer or at risk of developing aggressive prostate cancer comprises a subject with prostate cancer with a high Gleason score.

8. The method of claim 2, wherein the aggressive prostate cancer-related molecules further comprise CAMKK2 and PSA, wherein the subject with aggressive prostate cancer or at risk of developing aggressive prostate cancer comprises a subject at risk for post-surgical distant metastatic prostate cancer and/or biochemical recurrent prostate cancer.

9. (canceled)

10. A method of treating a subject with aggressive prostate cancer or at risk of developing aggressive prostate cancer, comprising:
administering at least one of surgery, radiation, hormone therapy, chemotherapy, brachytherapy, cryotherapy, ultrasound, bisphosphate therapy, biologic therapy, or vaccine therapy to the subject having aggressive prostate cancer or at risk of developing aggressive prostate cancer, thereby treating the subject,
wherein prior to the administration of the at least one of surgery, radiation, hormone therapy, chemotherapy, brachytherapy, cryotherapy, ultrasound, bisphosphate therapy, biologic therapy, or vaccine therapy, the expression level of the aggressive prostate cancer-related molecules in a sample from the subject was determined to be increased as compared to a control, wherein the control represents expression of the aggressive prostate cancer-related molecules expected in a sample from a subject who does not have aggressive prostate cancer; and
wherein the aggressive prostate cancer-related molecules comprise folate hydro lase 1 (FOLH1), secreted protein acidic and rich in cysteine (SPARC), transforming growth factor beta 1 (TGFB1), calcium/calmodulin dependent protein kinase kinase 2 (CAMKK2), and prostate-specific antigen (PSA).

11. (canceled)

12. The method of claim 2, wherein the control comprises a control representing expression for the aggressive prostate cancer-related molecule(s) expected in a sample from a subject who does not develop post-surgical biochemical recurrent prostate cancer.

13. The method of claim 2, wherein the control comprises a control representing expression for the aggressive prostate cancer-related molecule(s) expected in a sample from a subject who does not develop post-surgical distant metastatic prostate cancer.

14. The method of claim 2, wherein the control comprises a control representing expression for the aggressive prostate cancer-related molecule(s) expected in a sample from a subject who does not have prostate cancer with a high Gleason score or a high grade group.

15. The method of claim 2, wherein the sample comprises a prostatectomy sample, a biopsy sample, blood sample, urine, semen, or expressed prostatic secretion sample.

16. The method of claim 15, wherein the sample is a prostatectomy sample.

17. The method of claim 15, wherein the sample is a formalin-fixed paraffin-embedded (FFPE) sample.

18. The method of claim 2, wherein the expression is determined using relative protein abundance or relative peptide abundance.

19. The method of claim 18, wherein the abundance is determined using mass spectrometry or immunohistochemistry assay.

20. The method of claim 19, wherein the mass spectrometry is selected reaction monitoring (SRM).

21. The method of claim 19, wherein the abundance is determined using a high-pressure, high-resolution separations coupled with intelligent selection and multiplexing (PRISM)-SRM assay.

22. The method of claim 2, wherein the subject has had a prostatectomy.

23. The method of claim 2, wherein the subject has had a prostatectomy, and the method further comprises post-treatment surveillance.

24. The method of claim 2, wherein the aggressive prostate cancer therapy comprises at least one of surgery, radiation, hormone therapy, chemotherapy, brachytherapy, cryotherapy, ultrasound, bisphosphate therapy, biologic therapy, or vaccine therapy.

25. A method, comprising:
treating a sample obtained from a subject with a protease, thereby forming a digested sample; and measuring expression of:
(a) folate hydrolase 1 (FOLH1), secreted protein acidic and rich in cysteine (SPARC), and transforming growth factor beta 1 (TGFB1);
(b) FOLH1, SPARC, TGFB1, and calcium/calmodulin dependent protein kinase kinase 2 (CAMKK2);
(c) FOLH1, SPARC, TGFB1, CAMKK2, EGFR, and NCOA2;
(d) FOLH1, SPARC, TGFB1, CAMKK2, and PSA; or
(e) FOLH1, SPARC, TGFB1, and PSA;
in the digested sample using mass spectrometry.

26. The method of claim 2, wherein the subject has had a prostatectomy, and the method further comprises post-treatment surveillance.

27. The method of claim 2, wherein the subject is human.
